(19) United States
(12) Patent Application Publication
(10) Pub. No.: US 2020/0126094 A1
(43) Pub. Date: Apr. 23, 2020

(54) MEDICAL RESEARCH FRAUD DETECTION SYSTEM AND SOFTWARE

(71) Applicant: BioIDC, Inc., Glendale, CA (US)

(72) Inventor: Sherif Mustafa ElHarazi, Glendale, CA (US)

(73) Assignee: BioIDC, Inc., Glendale, CA (US)

(21) Appl. No.: 16/658,045

(22) Filed: Oct. 19, 2019

Related U.S. Application Data
(60) Provisional application No. 62/810,405, filed on Feb. 26, 2019.
(60) Provisional application No. 62/747,674, filed on Oct. 19, 2018.

(51) Int. Cl. G06Q 30/00; G16H 50/70; A61B 5/1172

(52) U.S. Cl. CPC G06Q 30/0185; A61B 5/1172; G16H 50/70

(57) ABSTRACT

A method of detecting fraudulent patient behavior in a clinical research study includes receiving, at a computing device, a patient fingerprint image, from an imaging device of administrator or investigator computing device; comparing, at the computing device, the received patient fingerprint image to a database of existing patient fingerprint images to determine if the patient is previously enrolled in a clinical study; communicating a message identifying the patient may be eligible to enroll if no match is found between the received fingerprint image and the database of existing patient fingerprint images; and performing additional analysis with respect to the patient if a match is found between the received fingerprint image and the database of existing fingerprint images.

### RELATED APPLICATIONS

This application claims priority to application Ser. No. 62/747,674, filed Oct. 19, 2018, titled “Medical Research Fraud Detection System And Software,” and also claims priority to application Ser. No. 62/810,405, filed Feb. 26, 2019, titled “Medical Research Fraud Detection System and Software February 2019,” the disclosures of which are both hereby incorporated by reference.

### BACKGROUND OF THE INVENTION

Clinical research sponsors invest large amounts of resources and money on clinical trials in order to have drugs and/or procedures approved by governmental agencies. Clinical research sponsors pay patients to participate in such research studies. Unfortunately, some patients have decided to attempt to enroll in the same study at multiples sites (in order to collect additional payments); to enroll in research studies without waiting a mandatory waiting period after completing a prior research study; or to enroll in multiple research studies at the same time. In many cases, such patients utilize fake names or social security numbers in order to trick the system and obtain additional payments from research sponsor. There is no system currently in place to catch such patients who are committing fraud with respect to research sponsors.

### BRIEF DESCRIPTION OF THE DRAWINGS

FIG. 1 illustrates a flowchart for operation of a medical research fraud detection system according to embodiments;

FIG. 2A illustrates a medical research fraud detection computing system according to embodiments;

FIG. 2B illustrates a medical research fraud detection computing device server according to embodiments;

FIG. 3 illustrates a create and manage medical clinical study or trial process according to some embodiments;

FIG. 4A illustrates a patient screening or validation process in a medical research fraud detection system or software application according to some embodiments;

FIG. 4B illustrates a process for determining whether a patient is determined to be high risk according to some embodiments;

FIG. 4C illustrates a patient screening or validation process in a medical research fraud detection system or software application when fingerprint validation is not available according to some embodiments

FIG. 5A illustrates a process of sponsor activation in the medical clinical trial fraud detection software system according to some embodiments;

FIG. 5B illustrates a process of user authentication in a medical research fraud detection system according to some embodiments;

FIG. 6 illustrates a process for initiating or creating a clinical study or clinical trial in the medical research fraud detection system according to some embodiments;

FIG. 7 illustrates a clinical trial or study site management process according to some embodiments;

FIG. 8 illustrates a clinical study site assignment process according to some embodiments;

FIG. 9 illustrates a medical research fraud detection system financial management process according to some embodiments;

FIG. 10 illustrates an auditing process in the medical research fraud detection system according to some embodiments;

FIG. 11A illustrates a sponsor administrator dashboard in a medical research fraud detection software application according to some embodiments;

FIG. 11B illustrates a CRO dashboard in a medical research fraud detection software application according to some embodiments;

FIG. 11C illustrates an investigator dashboard in a medical research fraud detection software application according to some embodiments;

FIG. 11D illustrates a study coordinator dashboard in a medical research fraud detection software application according to some embodiments; and

FIG. 11E illustrates a software administrator (e.g., IDC administrator) dashboard in a medical research fraud detection software application according to some embodiments

### DETAILED DESCRIPTION OF THE INVENTION

Described herein is a medical research fraud detection system to keep patients from committing fraud and/or obtaining excess payments from medical research sponsors. In embodiments, computer-readable software may be stored in memory devices of computing devices such as application servers and/or database servers and may be executed by one or more processors of the computing devices to perform the functions of the medical research fraud detection software. In embodiments, the computing devices (e.g., application servers and/or database servers) may be cloud-based servers and run by third parties such as Amazon. In embodiments, the computing devices (e.g., application servers and/or database servers) may be controlled by research sponsors and located at computing facilities controlled by the research sponsor and/or creator of the medical research fraud detection software. In some embodiments, computing devices located at research sponsor sites, investigator sites or CRO physical sites may include medical research fraud detection application software installed in memory devices of these computing devices and interface or communicate with the medical research fraud detection software installed on, for example, the medical research fraud detection system application servers and/or database servers. In some embodiments, individuals at CROs or research sponsor facilities may utilize computing devices such as mobile communication devices, tablets, laptops and/or desktop computers to interface with and/or communicate with the medical research fraud detection system application servers and/or database servers. In some embodiments, computing devices at investigator sites may include medical research fraud detection application software installed in memory devices of these computing devices and may interface or communicate with the medical research fraud detection software installed on the medical research fraud detection system application servers and/or database servers. In some embodiments, individuals at investigator sites may utilize computing devices such as mobile communication devices, tablets, laptops and/or desktop computers to interface with and/or communicate with the medical research fraud detection system application servers and/or database servers. In some embodiments, computing devices at administrator or other affiliated sites may include medical research fraud detection application software installed in memory devices of these computing devices and may interface or communicate with the medical research fraud detection software installed on the medical research fraud detection system application servers and/or database servers. In some embodiments, individuals at administrator sites may utilize computing devices such as mobile communication devices, tablets, laptops and/or desktop computers to interface with and/or communicate with the medical research fraud detection system application servers and/or database servers. No prior system had a coordinated network of medical research fraud detection system application servers and/or database servers that can interface with sponsor administrator, CRO administrator, software developer personnel, investigator or site administer computing devices so that patients at different sites could not try to double enroll, enroll too early, or exit studies after initial payments and try to enroll with other studies. In addition, the medical research fraud detection application servers and/or database servers may interface with a large number or research sponsors and thus be able to track when patients are attempting to engage in fraudulent behavior with multiple research sponsors.

In some embodiments, research patients or study patients do not have access to the medical research fraud detection system application servers and/or database servers. This is necessary because although it may be these patients' HIPAA medical data, the patients cannot have access to the medical research studies or protocols, research study data, and/or other patients' HIPAA data. The computing devices utilized at the research sponsor sites, the administrator sites, the CRO sites and/or the investigator sites may be client software applications that interface with the medical research fraud detection system application servers and/or database servers. In embodiments, the computer-readable instructions stored on the devices may be executable by one or more processors on these devices. In addition, in some embodiments, only an application programming interface with minimal software may be installed on computing devices at the administrator, investigator, research sponsor or CRO sites. In some embodiments, additional software modules may also be installed on the computing devices at the administrator, investigator, research sponsor and/or CRO sites in order to speed up the interaction with the medical research fraud detection system application servers and/or database servers. In some embodiments, the computing devices at the administrator, investigator, research sponsor or CRO sites may communicate with the medical research fraud detection application servers and/or database servers via wired and/or wireless communication protocols and the computing devices may be installed on global communication networks, personal area networks, wide area networks and/or local area networks. In some embodiments, due to the sensitive, private and/or personal nature of the data, the computing devices at administrator, investigator, research sponsor or CRO may be required to use multi-factor authentication and/or other authentication protocols in order to communicate with the medical research fraud detection software application servers and/or database servers. In some cases, the sensitive and private nature of the data may require that administrators, investigators, research sponsors, or CROs provide biometric identifiers to verify they are authorized or authenticated to use the medical research fraud detection system. In some embodiments, the application herein may refer to fraud detection application software, medical research fraud detection application software and/or clinical study fraud detection software which may be utilized interchangeably and/or may refer to the software installed on the medical research fraud detection application servers and/or database servers as well as the APIs and/or software installed on the administrator, investigator, research sponsor or CRO computing devices.

The foregoing, and other features and advantages of the invention, will be apparent from the following, more particular description of the preferred embodiments of the invention, the accompanying drawings, and the claims. In the following detailed description, numerous specific details are set forth to provide a thorough understanding of claimed subject matter. For purposes of explanation, specific numbers, systems and/or configurations are set forth, for example. However, it should be apparent to one skilled in the relevant art having benefit of this disclosure that claimed subject matter may be practiced without specific details. In other instances, well-known features may be omitted and/or simplified so as not to obscure claimed subject matter. While certain features have been illustrated and/or described herein, many modifications, substitutions, changes and/or equivalents may occur to those skilled in the art. It is, therefore, to be understood that appended claims are intended to cover any and all modifications and/or changes as fall within claimed subject matter.

References throughout this specification to one implementation, an implementation, one embodiment, some embodiments, embodiments, an embodiment and/or the like means that a particular feature, structure, and/or characteristic described in connection with a particular implementation and/or embodiment is included in at least one implementation and/or embodiment of claimed subject matter. Thus, appearances of such phrases, for example, in various places throughout this specification are not necessarily intended to refer to the same implementation or to any one particular implementation described. Furthermore, it is to be understood that particular features, structures, and/or characteristics described are capable of being combined in various ways in one or more implementations and, therefore, are within intended claim scope, for example. In general, of course, these and other issues vary with context. Therefore, particular context of description and/or usage provides helpful guidance regarding inferences to be drawn.

Likewise, in this context, the terms “coupled”, “connected,” and/or similar terms are used generically. It should be understood that these terms are not intended as synonyms. Rather, “connected” is used generically to indicate that two or more components, for example, are in direct physical, including electrical, contact; while, “coupled” is used generically to mean that two or more components are potentially in direct physical, including electrical, contact; however, “coupled” is also used generically to also mean that two or more components are not necessarily in direct contact, but nonetheless are able to co-operate and/or interact. The term “coupled” is also understood generically to mean indirectly connected, for example, in an appropriate context.

The terms, “and”, “or”, “and/or” and/or similar terms, as used herein, include a variety of meanings that also are expected to depend at least in part upon the particular context in which such terms are used. Typically, “or” if used to associate a list, such as A, B or C, is intended to mean A, B, and C, here used in the inclusive sense, as well as A, B or C, here used in the exclusive sense. In addition, the term “one or more” and/or similar terms is used to describe any feature, structure, and/or characteristic in the singular and/or is also used to describe a plurality and/or some other combination of features, structures and/or characteristics.

Likewise, the term “based on,” “based, at least in part on,” and/or similar terms (e.g., based at least in part on) are understood as not necessarily intending to convey an exclusive set of factors, but to allow for existence of additional factors not necessarily expressly described. Of course, for all of the foregoing, particular context of description and/or usage provides helpful guidance regarding inferences to be drawn. It should be noted that the following description merely provides one or more illustrative examples and claimed subject matter is not limited to these one or more illustrative examples; however, again, particular context of description and/or usage provides helpful guidance regarding inferences to be drawn.

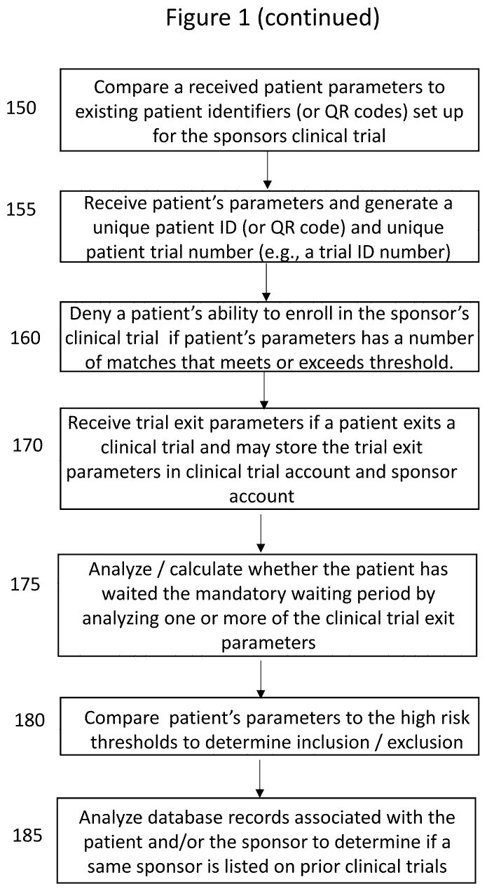

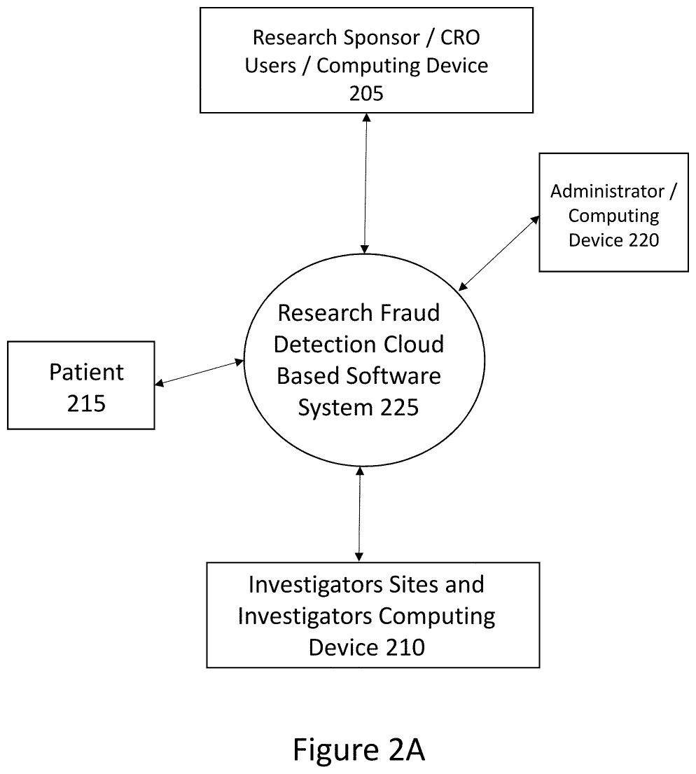

FIG. 2A illustrates a medical research fraud detection computing system according to embodiments. FIG. 2B illustrates a medical research fraud detection computing device or server according to embodiments. In embodiments, a medical research fraud detection system 200 may comprise a research sponsor/clinical research organizer (CRO) computing device 205, an administrator computing device 220, a patient or computing device utilized by a patient 215, an investigator site or investigator's computing device 210, and/or a research fraud detection cloud based server 225. In some embodiments, the research fraud detection computing device 225 may be one or more server computing devices, one or more application server computing devices and/or one or more database server computing devices and/or may not be a cloud-based server or computing device. In embodiments, the administrator computing device 220, the research sponsor computing device 205, the computing device utilized by a patient 215, and an investigator's computing device 210 may communicate with the research fraud detection cloud-based server via a wireless communication network or a wired communication network. Although one computing device is illustrated, each of the sites may include multiple computing devices. In embodiments, the communication networks may include global communications networks (e.g., the Internet), local area networks, private or proprietary networks, and/or intranets. In embodiments, a research fraud detection cloud based server 225 may include one or more medical research fraud detection application servers 255 and/or one or more medical research fraud detection database servers 260.

In embodiments, an individual at a research sponsor or clinical research organization may utilize a sponsor computing device 205 to communicate with the research fraud detection cloud based server 225 to create new research studies, to assign or invite investigator sites and/or to manage and keep track of users. In embodiments, the sponsor computing device 205 may communicate with the research fraud detection cloud based server 225 to accept or reject patients and/or to view patient history information.

In embodiments, an individual or professional at an investigator site may utilize an investigator computing device 210 to communicate with the research fraud detection cloud based server 225 intake and assign site users, to enter patient-related information, and/or to access research studies. In embodiments, an individual or professional at an investigator site may utilize an investigator computing device 210 to communicate with the research fraud detection cloud based server 225 to enroll patients in a research study, to view or review patient data and/or patient history, to view research study data or information, to generate unique ID numbers for a patient and/or to generate patient QR codes.

In embodiments, a patient or user cannot use any computing device 215 to directly access a research fraud detection server 225. In embodiments, a patient may use a computing device 215 to provide biometric data to provide proof of identify and verify that the patient is not gaming the system or committing fraud. In embodiments, computing device 215 may include a retinal scanner, a fingerprint scanner, an imaging device, a microphone and/or voice recognition software to verify the user's identity in order for the patient to be verified and/or for patient data to be included in the research study.

In embodiments, a user may be assigned a role of administrator and may utilize an administrator computing device to perform administrative tasks for the research fraud detection server 225. In embodiments, an administrator or super user may be able to add and/or remove sponsors, CROs, investigative sites, patients, users, and/or research studies. In some embodiments, an administrator or super users may not have access to HIPAA-regulated data.

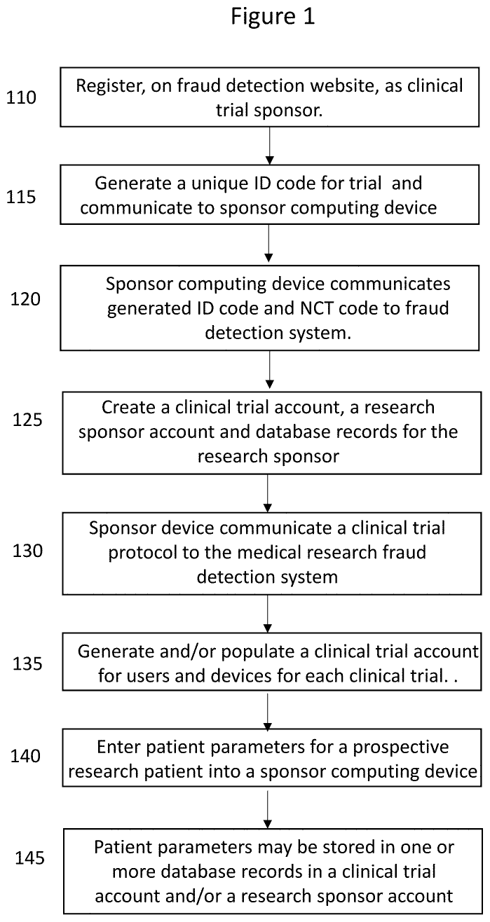

FIG. 1 illustrates a flowchart for operation of a medical research fraud detection system according to embodiments. In some embodiments, a research sponsor computing device may login to a medical research fraud detection web site (e.g., a medical research fraud detection cloud-based server) and register 110 as a research sponsor in a medical research fraud detection system. In embodiments, medical research fraud detection software may generate a unique identification (ID) code and communicate 115 the unique ID code to the research sponsor computing device to identify a research sponsor.

In embodiments, a research sponsor computing device may register specific research studies with a medical research fraud detection system. In embodiments, the research studies may be associated with and/or be affiliated with a National Clinical Trial (NCT) identifier value. In embodiments, for example, a unique ID code may be an NCT number generated and/or issued by www.clinicaltrials.gov. In embodiments, the research sponsor computing device may communicate a unique ID code as well as a clinical trial identifier value (NCT identifier value) 120 to the medical research fraud detection computing device or server in the medical research fraud detection system.

In embodiments, an administrator may utilize the medical research fraud detection software to create 125 a clinical trial account, a research sponsor account and database records for the research sponsor in the medical research fraud detection system (e.g., the accounts and/or records may be created and/or established in the medical research fraud detection database server 260). In embodiments, a user or individual affiliated with the sponsor or CRO may utilize the medical research fraud detection software to create a clinical trial account, a research sponsor account and/or database records for the research sponsor. In embodiments, the clinical trial account, the research sponsor account and database records may be stored in one or more memory devices of the medical research fraud detection cloud-based software system. In embodiments, one or more database servers 260 may include the one or one more memory devices that store the clinical trial account, the research sponsor account and the associated database records.

In embodiments, the research sponsor computing device 205 may communicate 130 a clinical trial protocol to the medical research fraud detection system or server 225. In embodiment, the clinical trial protocol may comprise a NCT identifier, a clinical trial protocol number, and/or a study investigational product name, identifier or characteristic. In embodiments, an investigational product may refer to a preventative (e.g., vaccine), a therapeutic (e.g., a drug or biologic), a device, a diagnostic, or palliative used in a clinical trial. In embodiments, an investigational product may be an unlicensed product of a licensed produced when used or assembled (formulated or packaged) differently from the approved form, when used for an unapproved indication, or when used to gain further information about an approved use.

In embodiments, an individual from a research sponsor organization or a CRO may utilize the medical research fraud detection software to generate and/or populate 135 a clinical trial account for users and devices for each clinical trial. In embodiments, an administrator or super user may utilize the medical research fraud detection software to generate and/or populate 135 a clinical trial account for users and devices for each clinical trial. In embodiments, the clinical trial account may include one or more database records that identify usernames and passwords for all users as well as computing device information (e.g., computing device name and/or IP address) for all computers and/or sites associated with the clinical trial. In embodiments, the creation of this clinical trial account prevents unauthorized users, staff and/or sites from accessing this clinical trial and/or the medical research fraud detection software. In embodiments, portions of the medical research fraud detection software may be executable by processors on the research sponsor or CRO computing device 205 and portions of the medical research fraud detection software may be executable by processors on the medical research fraud detection server 225 (e.g., or application server 255).

A patient may then enroll through an employee of a clinical sponsor who has a computing device 215 such as a tablet. In embodiments, an employee at an investigator site may utilize an investigator site computing device 210 to enroll in an established clinical trial. In embodiments, an employee of a clinical trial sponsor whose clinical trial has been registered with the medical research fraud detection software may enter patient parameters 140 for a prospective research patient into a sponsor computing device and the sponsor computing device may be communicated to the medical research fraud detection system and software. In embodiments, if this is the first time the medical research fraud detection system is being utilized for a specific clinical trial, then the prospective patient parameters may be stored 145 in one or more database records in a clinical trial account and/or a research sponsor account in the medical research fraud detection system (e.g., in the medical research fraud detection database server 260). In embodiments, the patient parameters may be a patient's last name, a patient's first name, a patient's date of birth and/or a last four numbers of the patient's social security number. In embodiments, the patient's parameters may be an ID number or driver's license number, a sex at birth parameter, a race or ethnicity parameters and/or a city of residence parameters. In embodiments, a clinical trial sponsor computing device 205 (or an investigator site's computing device 210) may comprise a fingerprint scanner and a patient may scan their fingerprint, and an image of the fingerprint may be communicated to the medical research fraud detection system or server 225. In embodiments, thus, the patient parameters may be an image of the patient's fingerprint. In embodiments, a clinical trial sponsor computing device may comprise one or microphones and a patient may be asked to speak a provided phrase (e.g., such as “medical research is awesome”). In embodiments, the one or more microphones may capture the spoken phrase, convert it to an audio file, and then the clinical sponsor computing device may communicate the captured audio file to the medical research fraud detection system or server 225. In embodiments, the patient's parameter may include a voice recognition audio file.

In embodiments, specific parameters may be referred to as absolute or significant parameters. In embodiments, the medical research fraud detection software matching two or more absolute parameters may be an indication that the patient is engaging in fraud by attempting to enroll more than once in a clinical trial. In embodiments, for example, absolute and/or significant parameters may be: 1) patient last name; 2) date of birth; 3) last 4 numbers of social security number; 4) a fingerprint image; and/or 5) a voice audio file. In embodiments, if the medical research fraud detection software determines a match with two or more absolute parameters, this may be an indication that the patient is engaging in fraudulent behavior.

Fraud Detection for Same Trial—

In embodiments, once a sponsor computing device 205 or an investigator's site computing device 210 communicates the patient's parameters to the medical research fraud detection system 225, the medical research fraud detection software may compare 150 the received patient parameters to existing or unique patient ID number (or QR codes) set up for the sponsors clinical trial. In embodiments, the medical research fraud detection software may compare 150 the received patient parameters to existing patient ID numbers. In embodiments, if there is less than a predetermined number of matches of the patient parameters to patient parameters associated with and/or corresponding to existing QR codes (or existing patient ID numbers), then the medical research fraud detection software and system 225 may continue on to step 155 and create a QR code (or a unique patient ID number) for what is determined to be a new patient in the clinical study.

In embodiments, the medical research fraud detection software or system 225 may receive the patient's parameters and may generate 155 a QR code (or unique patient ID number) and a unique patient trial number (e.g., an trial ID number) for the patient. In embodiments, the QR code (or unique patient ID) and the unique patient trial number may be stored in one or more memory devices (e.g., one or more database records in one or more database servers 260 in the research fraud detection software or system 225). In embodiments, the QR code (or unique patient ID number) and patient trial number may be stored in one or more records in database records in a sponsor clinical trial account and/or a global sponsor account (e.g., in, for example, the medical research fraud detection database server 260), and thus may not only be limited to the account setup for a specific trial. In embodiments, a QR code (or unique patient ID) generated by the medical research fraud detection software or system 225 may include the patient's parameters, a NCT protocol number, a date of enrollment in the trial, and, at a later time, a date of exit of the patient from the clinical trial.

In embodiments, once the unique patient ID (or QR code) is generated, the medical research fraud detection software or system 225 may not allow absolute or significant parameters to be modified or changed. If a patient (or a clinical sponsor and/or clinical sponsor computing device) tries to modify absolute or significant parameters, the medical research fraud detection software or system 225 may generate an error message and communicate the error message to a sponsor computing device 205, an investigator computing device 210 and/or an administrator or super user computing device 220. In embodiments, an error message may also communicate a message to a medical research fraud detection administrator's computing device 220. In embodiments, the medical research fraud detection software may also lock the patient's records in the clinical trial account and/or research sponsor account, and/or prevent any more interaction or communication with the patient (or any computing device associated with the patient).

In embodiments, the medical research fraud detection software or system 225 may also transmit an informed consent form to the sponsor computing device 205 and/or the investigator computing device 210 or computing device being provided to patient 215 to have the patient electronically sign the document. In embodiments, the sponsor computing device 205, investigator computing device 210 or other computing devices 215 may communicate the signed informed consent form to the medical research fraud detection software for storage in one or more memory devices associated with the patient. In embodiments, the signed informed consent form may be stored in a clinical trial account and/or the sponsor account. In embodiments, the medical research fraud detection software or system 225 may also include the NCT protocol number and a date of screening in the patient's database records (e.g., which may be stored in the medical research fraud detection software or system 225.

In embodiments, if the medical research fraud detection software or system 225 determines that the received patient parameters as compared to the existing patient parameters (e.g., associated with the unique patient ID or QR codes), if the exact number of predetermined matches is equal to or is greater than a predetermined matches threshold, then the medical research fraud detection software or system 225 may deny 160 a patient's ability to enroll in the sponsor's clinical trial and/or also may end the patient's prior participation in the sponsor's clinical trial (because if there are matches, then this means the patient has enrolled in clinical trials previously). In embodiments, the medical research fraud detection software or system 225 may generate an error message and communicate an error message to the sponsor computing device 205, the investigator computing device 210 and/or a computing device being provided to the patient 215. In embodiments, the employee may then inform the patient of the duplicate enrollment and request an explanation from the patient as to why there is duplicate enrollment. In embodiments, the medical research fraud detection software or system may also lock the patient's existing unique ID code (or QR code) and/or database records (e.g., in the medical research fraud detection software database server 260) to prevent the patient from participating in the clinical trial unless a satisfactory explanation is provided by the patient. In that case, a system administrator may then unlock the patient's existing unique ID number (e.g., or QR code) and/or database records. In embodiments, the predetermined number of matches of parameters or absolute parameters (e.g., a threshold) may be three. This may be because it is possible for a patient to have a same last name, a same birthdate, but not to have a match of the third absolute parameter of a social security number, for example). In embodiments, sometimes, if the medical research fraud detection software or system 225 finds a match with an existing fingerprint image (and/or a voice recording) in an existing unique patient ID (or QR record), the medical research fraud detection software or system 225 may deny a patient entry into the clinical trial. This is because these patient parameters or characteristics are so unique that if there is a match, then it is very likely that the patient is engaging in fraudulent behavior.

In embodiments, patients may also try to enroll in medical research studies too early after exiting and/or finishing a study, which may be fraudulent behavior as well, that leads to erroneous test results. It is thus vital for a medical research fraud detection software and system 225 to keep track of timing of entering and exiting into clinical trials (as well as reasons for exiting clinical trials), as well as what types of clinical trials a patient has previously enrolled in. In embodiments, the medical research fraud detection software or system 225 may receive 170 trial exit parameters if a patient exits a clinical trial and may store the trial exit parameters in one or more memory devices of the medical research fraud detection system or software (e.g., database records in a clinical trial account and/or sponsor account of a medical research fraud detection database servers). In embodiments, patients may exit a clinical study for a number of reasons. In embodiments, a trial patient exit parameters may include a date of exit (extremely important for determining future eligibility for clinical trials), as well as reasons for exit. In embodiments, reasons for exit from a clinical trial may be: (a) screening failure at clinical trial site, e.g., patient inclusion criteria not met or patient exclusion criteria met; (b) lost to follow up (patient did not follow clinical trial regime whether taking medication and/or reporting to trial site for follow up); (c) withdrawn consent to clinical trial; (d) withdrawn from clinical trial because internal or external investigator has determined non-compliance with clinical trial and/or safety risk if further participating in clinical trial; (e) the clinical trial sponsor or Independent Research Board (IRB) has withdrawn patient from clinical trial; (f) the study has been terminated by the sponsor; and/or (g) the clinical trial has been completed.

In embodiments, the medical research fraud detection software or system 225 may also evaluate and/or detect fraud or non-compliance across a number of studies from a number of sponsors. In embodiments, for example, a patient may try to reenroll in a future clinical trial program, but may not have waited a mandatory waiting period (e.g., 30 days is one waiting period and other programs may have other mandatory waiting periods). Therefore, once a patient enrolls in a clinical trial and has passed the introductory screening (e.g., verifying that there are no matches of absolute or significant parameters in the clinical trial in which the patient is attempting to enroll), the medical research fraud detection software or system 225 may analyze and/or calculate 175 whether the patient has waited the mandatory waiting period by analyzing one or more of the clinical trial exit parameters for patients exiting a medical research study. If the medical research fraud detection software or system determines that a mandatory waiting period has been met, the patient may be allowed to enroll in the current clinical trial. If the medical research fraud detection software or system 225 determines that the mandatory waiting period has not been met, the patient may be prevented from entering the clinical trial and the medical research fraud detection software or system 225 may communicate a message to the sponsor computing device 205, the investigator site computing device 210 and/or an administrator computing device 220 providing the reason for rejection from the clinical trial. In addition, the medical research fraud detection software or system 225 may update fields of records in the medical research fraud detection system memory devices or database servers 260 (e.g., may update database records to identify that patient tried to enroll too quickly after prior clinical trial or clinical trial exit).

High Risk Patients—An additional feature of the proposed medical research fraud detection software or system is the ability to flag, identify and/or notice patients that are engaging in high-risk behavior that compromises the credibility and/or validity of clinical trial results. In embodiments, a high-risk patient may be a patient that has engaged in multiple studies at once, withdraws consent from trials multiple times, or is lost to follow up or other similar reasons. In embodiments, if the medical research fraud detection software or system determines that a patient is engaged in high-risk activities or behaviors, the medical research fraud detection software or system 225 may flag or identify the patient for a ban from enrolling in any clinical trial studies unless acceptable reasons have been received and verified by clinical trial sponsors or independent third party investigators. In embodiments, the medical research fraud detection software or system may identify the following high risk thresholds for clinical trials: 1) patient is lost to follow up in more than 2 studies during a 3 year period; 2) withdrawn consent by patient in more than 2 studies in the last calendar year; 3) withdrawn by third party investigator in more than one clinical trial study; 4) withdrawn by clinical trial sponsor or IRB in more than 1 clinical trial study for noncompliance; and/or 5) more than 3 screening failures (and thus exclusion from a clinical trial) in a 12 month timeframe. In embodiments, the medical research fraud detection software or system may compare 180 the patient's parameters to the high risk thresholds to determine or identify if the patient should be excluded from the clinical trial they are trying to enroll in and/or have exited from. If the medical research fraud detection software or system determines that a patient has met and/or exceeded the high risk threshold, the patient may be prevented from entering the clinical trial and/or the medical research fraud detection software or system may communicate a message to the sponsor computing device 205, the investigator site computing device 210 and/or the administrator or super user computing device 220 providing the reason for rejection from the clinical trial. In addition, the medical research fraud detection software 225 may update fields of records in the medical research fraud detection system memory devices or database server 260 (e.g., may update database records to identify that patient tried to enroll too quickly after prior clinical trial or clinical trial exit) and identify that further follow up is needed with respect to this patient. In embodiments, for example, the clinical client sponsor computing device 205, the investigator site computing device 210 and/or the administrator or super user computing device 220 may communicate with and/or contact the patient for follow up and/or may report the patient to regulatory and/or governmental organizations.

In embodiments, the medical research fraud detection software or system 225 may also analyze and detect whether a patient has tried to enroll in multiple studies by the same sponsor (which may not be allowed or authorized). In embodiments, after the medical research fraud detection system 225 has received the patient's parameters, the medical research fraud detection software or system 225 may run an additional check and determine if the patient has enrolled in other medical trials utilized by the same sponsor. In embodiments, the medical research fraud detection software or system 225 may analyze 185 database records (e.g., in the medical research fraud detection database server(s) 260) associated with the patient and/or the sponsor to determine if a same sponsor is listed on prior clinical trials. If the medical research fraud detection software or system 225 determines that a patient has not enrolled in a clinical trial by the same sponsor, the patient may be allowed to enroll in the current clinical trial. If the medical research fraud detection software or system 225 determines that the patient has enrolled in a clinical trial by the same sponsor (and that is forbidden by the inclusion criteria or identified in the exclusion criteria, the patient may be presented from entering the clinical trial and the medical research fraud detection software or system 225 may communicate a message to the sponsor computing device 205, investigator computing device 210 and/or administrator or super user computing device 220 providing the reason for rejection from the clinical trial. In addition, the medical research fraud detection software or system 225 may update fields of records in the medical research fraud detection system memory devices or database servers 260 (e.g., may update database records to identify that patient could not enroll due to prior enrollment by the same sponsor). In embodiments, different NCT numbers (for different clinical trials that are being held by the same sponsor) may be used as another check to verify that a patient is not violating rules and enrolling in studies that are run by the same sponsor. In embodiments, the medical research fraud detection software or system 225 may compare the patient's parameters against clinical trial identifier numbers in order to determine if the patient is engaging in unacceptable behavior and needs to be excluded from the current clinical trial the patient is trying to enroll in.

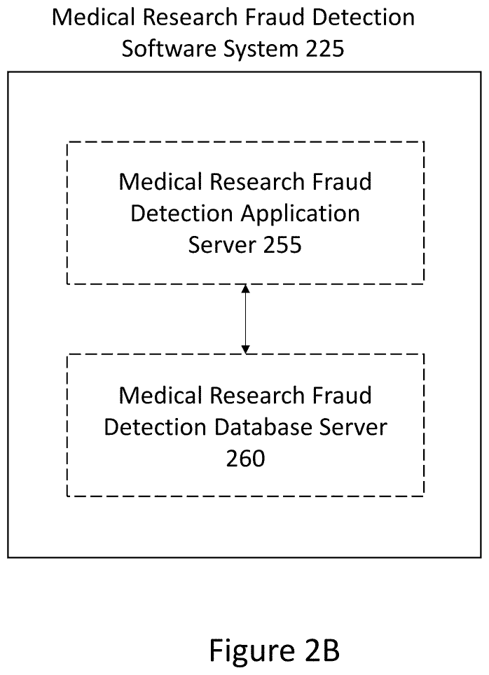

FIG. 3 illustrates a create and manage medical clinical study or trial process according to some embodiments. In some embodiments, the medical fraud detection system or software application performs the actions described in the processes described herein. In some embodiments, a sponsor may have set up a clinical trial. In some embodiments, the sponsor has identified and selected a number of sites that are able to administer the clinical trial. In some embodiments, the study coordinator, sponsor administrator, (or investigator) may utilize local computing devices to provide this information to the fraud detection software application. In some embodiments, for a specific site of the clinical trial, the medical fraud detection software application may receive the clinical trial or study details and/or parameters 305 for a new clinical trial or clinical study. In some embodiment, the clinical trial or study information or parameters display may be a study ID, a study type, a NCT number, a study description, a clinical indication, an age group for the trial or study, a gender type for the trial or study, and/or a race or ethnicity for the trial or study.

In some embodiments, the study coordinator (or an investigator) may add in a start date and/or end date as well as comments for the clinical trial or study. In some embodiments, the study coordinator (or investigator) may utilize local computing devices to provide this information to the fraud detection software application. In some embodiments, the fraud detection software application may receive the clinical trial or clinical study start date, the clinical trial end date and/or the clinical trial comments 310. In some embodiments, the study coordinator (or an investigator) may add in patients to trial. In some embodiments, the fraud detection software application may receive a request to add 315 in the patient to the clinical trial or study. In some embodiments, the patient may be added for a specific site.

In some embodiments, the fraud detection software application may verify the patient is enrollment eligible in the study utilizing fingerprint validation 320. In some embodiments, the fraud detection software application may verify the patient is enrollment eligible utilizing other biometric validation (including, but not limited to retinal scans, facial scan analysis, voice recognition and/or breath analysis). In some embodiments, the fraud detection software application may verify the patient is enrollment eligible in the study utilizing document validation or document parameters validation. In this embodiment, data and/or parameters are received based upon a patient's verifiable governmental documents (e.g., driver's license, passport, social security card and/or other verifiable documents.

In some embodiments, if the fraud detection software application verifies the patient is enrollment eligible, the fraud detection software application may verify that the patient is not high risk 325 and may allow the patient to move to registration if the patient is not determined to be or identified as high risk.

In some embodiments, if the patient is determined to be high risk, the fraud detection software application may communicatee with a study administrator or study administrator's computing device to determine whether or not the rejection of the patient as high risk is overridden by the study administrator and/or administrator computing device. In some embodiments, the fraud detection software application may receive authorization to enroll the high-risk patient 330.

In some embodiments, the patient may proceed with registration. In some embodiments, the fraud detection software application may receive registration parameters and/or information from the eligible patient 335. In some embodiments, the registration parameters may include, but not be limited to, first name, middle name, last name, date of birth, gender affiliation, drivers license (or other governmental identification parameters), last four numbers of social security number, race or ethnicity, city, state and/or country.

In some embodiments, the patient may also be requested to provide a voice sample or voice file by speaking into a microphone in the study administrator's computing device. In some embodiments, the fraud detection software application may receive a patient's voice file 340.

In some embodiments, the patient may also need to consent to the clinical study and the parameters, rules and/or protocols of the clinical study. In some embodiments, the fraud detection software application may receive a patient's consent indicator along with an image of the patient's signed consent document 345.

In some embodiments, the actual identity of the patient should be hidden from many users of the system in order to protect the anonymity of the patient. This may be done by generating a unique identifier for the patient. In some embodiments, the fraud detection software application may generate a QR code for the patient 350. In some embodiments, the QR code may include the patient's name, date-of-birth, last 4 digits of social security number, governmental identifier number and/or race/ethnicity or values representative of these parameters. In some embodiments, after the QR code is generated, the patient information may be marked or classified as read-only and thus may no longer be able to be edited and/or updated. In some embodiments, the fraud detection software application may create a QR code for each study a patient has enrolled in. For example, the patient may have a QR code for an older clinical trial, waited the specified timeframe and a different QR code for a current clinical trial. In some embodiments, this may complete the patient registration process.

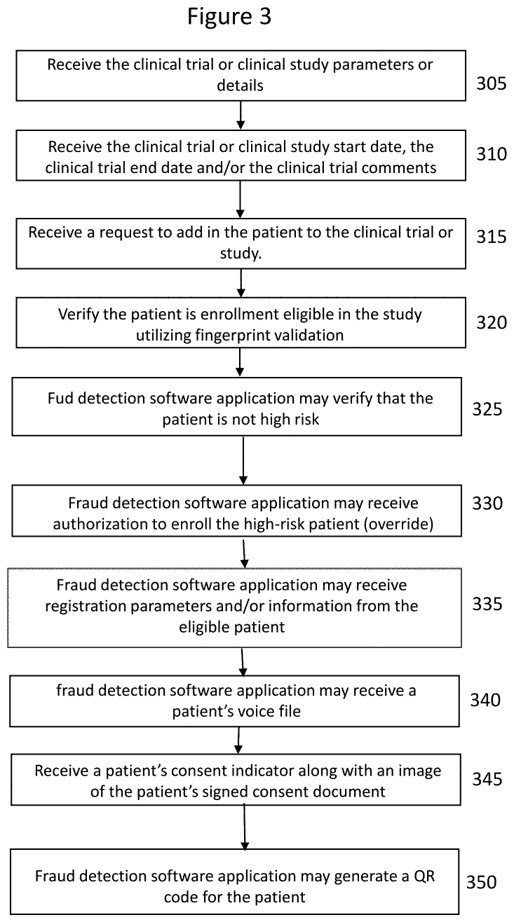

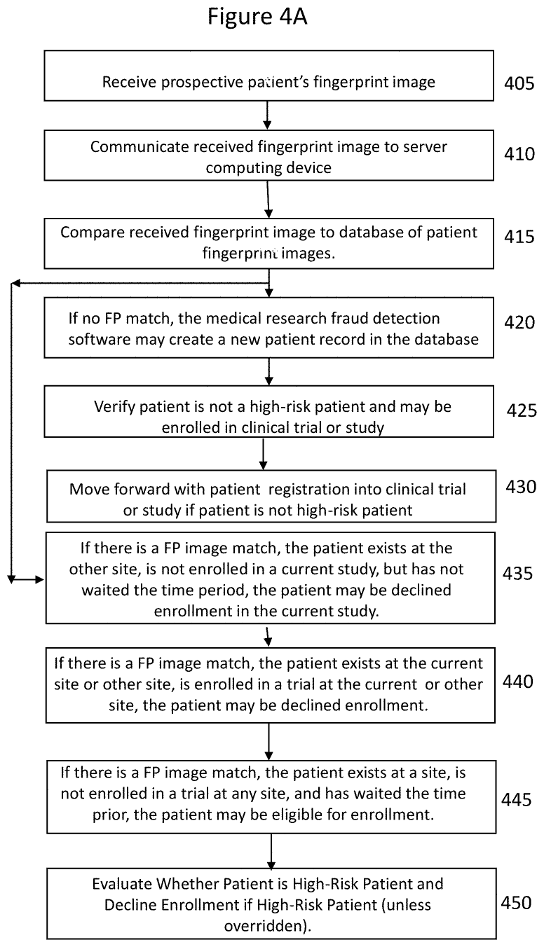

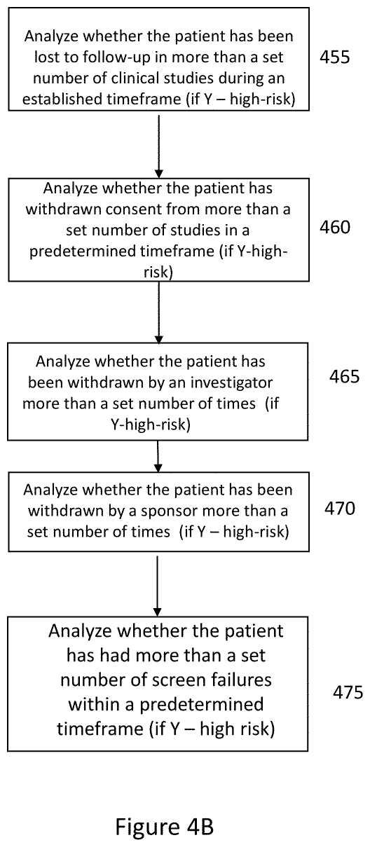

FIG. 4A illustrates a patient screening or validation process in a medical research fraud detection system or software application according to some embodiments. In some embodiments, a patient may want to enroll in a medical research study. In some embodiments, a patient may go to a site participating in the medical study and an operator may initiate a medical fraud detection software application on a computing device. In some embodiments, a patient's fingerprint may be captured at a remote computing device or local site computing device (e.g., a investigator's, CRO's, and/or sponsor's computing device) and the fingerprint image may be received 405 at this local site computing device. In some embodiments, the local site computing device may communicate 410 the received fingerprint image to a medical research fraud detection server. In some embodiments, computer-readable instructions executable by one or more processors of the fraud detection server computing devices may compare 415 the received fingerprint image of the patient to a database of fingerprint images for individuals or patients who have previously enrolled in medical research studies or trials. In some embodiments, this comparison process (and most of the other processes described herein) may be performed at the fraud detection cloud-based server, the fraud detection application server, and/or the fraud detection database server. If no match for the received patient fingerprint image iOs located, the medical research fraud detection software may create 420 a new patient record in the database. In some embodiments, a plurality of patient records may be stored in the patient database. In some embodiments, if the patient does not exist, a message may be generated by the fraud detection application software indicating the patient does not exist in the medical research fraud detection system and does the operator want to proceed to registering the new patient. In some embodiments, if there was no FP match, the fraud detection application software may determine if the patient is a high-risk patient 425 and deny enrollment if the patient is determined to be a high-risk patient. In some embodiments, if the fraud detection application software determines the patient is not a high-risk patient, the fraud detection application software may move forward 430 with the registration process.

In some embodiments, if there is a fingerprint image match, the patient exists at another site, the patient is not currently not undergoing any trial or study, but has not passed the predetermined time window, the patient may be declined enrollment in the current study or trial 435. In some embodiments, a message may be generated identifying “This Patient has not met 30 days window period between clinical trials. Patient eligible to enroll on [Exact Date]”.

In some embodiments, if there is a fingerprint image match, the patient exists at the other site or the current site, and the patient is currently undergoing a trial at the other or another Site, the fraud detection software may decline to enroll the patient in the current study or trial 440. In some embodiments, the fraud detection software may communicate a warning message, such as “The patient exists, and is currently enrolled in a clinical trial at another Site.” In some embodiments, the message may be communicated in a bright or identifying color (e.g., red color).

In some embodiments, if there is a fingerprint image match, the patient exists at the current site and is currently undergoing the trial at the current site, the fraud detection software may decline to enroll the patient in the current study or trial 440. In some embodiments, the fraud detection software application may communicate a warning message, “The patient exists, and is currently enrolled in a clinical trial at my Site.” In some embodiments, the message may be communicated in a bright or loud color (e.g., red color) to deny the enrollment.

In some embodiments, if there is a fingerprint image match, the patient exists at another site, or the current site, is not currently enrolled in a current trial, and has passed a predetermined time window, the patient may be eligible to be enrolled 445 in the current study or trial. In some embodiments, a message may be generated identifying “This Patient is present at some other Site or at this site, is currently not undergoing any trial, and has crossed 30 days window period after the Trial. Do you want to pull this patient to your Site, and add in the trial.”

In some embodiments, if the patient is eligible to be enrolled in the trial or study, the fraud detection software may evaluate the patient's record in the database to determine whether the patient is a high-risk patient. If the patient is determined to be a high-risk patient, the fraud detection software may deny the patient enrollment in the current trial or study 450. In some embodiments, an administrator or sponsor may override the denial of enrollment by the fraud detection software if they determine the high-risk patient may be in the current trial.

The ability to designate patients as high-risk patients for clinical studies based on previous actions is another unique aspect to the clinical trial fraud detection software application. There is no system that is able to obtain this information from the many sources (or computing devices) to determine whether or not a patient is a high risk to leave the clinical trial or study due to bad behavior or prior actions. In some embodiments, the medical research fraud detection software includes this feature for analyzing a patient's record and/or other records in the medical research fraud detection system in order to identify whether high-risk behavior has been exhibited in the past. In some embodiments, the fraud detection software application may analyze the electronic history of the patient's behavior in previous studies in order to determine if the patient is high-risk. In some embodiments, this determination is based on actions of patient as recorded in the patient records and/or clinical trial records, and not on medical history of the patient (in other words, they are not analyzing the patient's medical response to the trial; instead the system is focused on whether the patient followed protocol, completed the study, and/or exhibited professional behavior during the clinical study. FIG. 4B illustrates a process for determining whether a patient is determined to be high risk according to some embodiments. In some embodiments, for example, the fraud detection software application may analyze 455 whether the patient has been lost to follow-up in more than a set number of clinical studies during an established timeframe. In some embodiments, the medical research fraud detection software is analyzing whether the patient has completed studies and/or participated in all activities (or if they have for some reason left the clinical study). In some embodiments, the set number of studies may be two studies in more than a three-year period. In this embodiment, for example, the patient may not participate in all the events in the clinical study and thus may be deemed to be lost to follow-up.

In some embodiments, the fraud detection software application may analyze whether the patient has withdrawn consent from more than a set number of studies in a predetermined timeframe in order to determine whether the patient has been classified as high-risk 460. In some embodiments, the set number of studies may be two studies and the predetermined timeframe may be twelve months when analyzing whether or not the patient has been classified as high risk under this factor. This may be referred to or classified as “withdrawn consent by patient.”

In some embodiments, the fraud detection software application may analyze whether the patient has been withdrawn by an investigator more than a set number of times in order to determine whether the patient has been classified as high-risk 465. In some embodiments, the set number of studies may be one study where the patient has been withdrawn by an investigator. In some embodiments, a patient may be withdrawn by investigator for safety reasons or non-compliance (which may or may not be determined by the fraud detection software application).

In some embodiments, the fraud detection software application may analyze whether the patient has been withdrawn by a sponsor more than a set number of times in order to determine whether the patient has been classified as high-risk 470. In some embodiments, the set number off studies may be one study where the patient has been withdrawn by a sponsor. In some embodiments, a patient may be withdrawn by sponsor for not meeting inclusion and/or exclusion criteria.

In some embodiments, the fraud detection software application may analyze whether the patient has had more than a set number of screen failures within a predetermined timeframe to determine whether the patient has been classified as high risk 475. In some embodiments, the set number of screen failures may be 3 and the predetermined timeframe may be 12 months. In some embodiments, screen failures may refer to a patient signing a consent form and the fraud detection software application receiving this and then the patient not meeting and/or passing the inclusion and/or exclusion criteria.

FIG. 4C illustrates a patient screening or validation process in a medical research fraud detection system or software application when fingerprint validation is not available according to some embodiments. In some embodiments, a clinical study or trial site may not have access to fingerprint scanning either because he computing devices utilized there do not have fingerprint scanning or because there has been a malfunction of the fingerprint scanning system software and/or hardware. In these embodiments, a patient may be verified by an individual at the clinical trial or study site who reviews, evaluates and/or validates patient's documents. In some embodiments, the data, information and/or parameters may be entered into a computing device and communicated to the medical research fraud detection system or software application for validation. In this embodiment, the data, information and/or parameters received that are based on the verifiable governmental documents are compared to existing patient parameters, data and/or information in order to see if the patient has enrolled before.

In some embodiments, a patient may want to enroll in a medical research study or trial. In some embodiments, a patient may go to a site participating in the medical study and an operator may initiate a medical fraud detection software application on a computing device but there may not be fingerprint scanning capability and/or functionality. In some embodiments, the administrator and/or operator at the clinical or trial site may obtain patient's parameters, data and/or information from two or more verifiable sources. In some embodiments, the verifiable sources may be government-issued identification documents (e.g., Driver's licenses or other state photo identity cards issued by Department of Motor Vehicles (or equivalent); U.S. passport; U.S. passport card; DHS trusted traveler cards (Global Entry, NEXUS, SENTRI, FAST); U.S. Department of Defense ID, including IDs issued to dependents; Permanent resident card; Border crossing card; State-issued Enhanced Driver's License; federally-recognized; tribal-issued photo ID; HSPD-12 PIV card; Foreign government-issued passport; Canadian provincial driver's license or Indian and Northern Affairs Canada card; Transportation worker identification credential; U.S. Citizenship and Immigration Services Employment Authorization Card (I-766); and/or U.S. Merchant Mariner Credentials. In some embodiments, government-issued documents might not be available (although these documents are preferable). In these embodiments, other acceptable documents which may be verifiable are: Credit card; Acceptable proof of SSN documents (full SSN required) for commercial drivers; Social Security Card; Medicare card; U.S. Armed Forces Identification Cards: Active-DD 2, Retired-DD 2, Reserved-DD 2, Dependent-DD 173; Military separation document-DD 214; Social Security Administration (SSA) Account Card; W-2 form; SSA-1099 form; and/or Non-SSA-1099 form.

In some embodiments, a patient's data, information and/or parameters may be captured at a remote computing device or local site computing device (e.g., a investigator's, CRO's, and/or sponsor's computing device) and the patient's data, information and/or parameters may be received 480 at this local site computing device. In some embodiments, the local site computing device may communicate 481 the received patient's data, information and/or parameters to a medical research fraud detection server. In some embodiments, computer-readable instructions executable by one or more processors of the fraud detection server computing devices may compare 482 the received parameters, data and/or information of the patient to a database of parameters, data and/or information for individuals or patients who have previously enrolled in medical research studies or trials. In some embodiments, the patient parameters, data and/or information that are compared to the database of existing patient parameters may be first name, last name, DOB, ID/Drivers License/Passport number, and/or last 4 digits of social security number. In some embodiments, if there is a match of all of the above parameters, then the medical research fraud detection software system and/or software application may consider this a verifiable patient parameter match. In some embodiments, a verifiable patient parameter match may be identified if two or more of the above-identified patient parameters are matched to the existing patient parameters. In some embodiments, this comparison process (and most of the other processes described herein) may be performed at the fraud detection cloud-based server, the fraud detection application server, and/or the fraud detection database server. If no patient parameter match for the received patient parameters is found, the medical research fraud detection software may create 483 a new patient record in the database. In some embodiments, a plurality of patient records may be stored in the patient database. In some embodiments, if the patient does not exist, a message may be generated by the fraud detection application software indicating the patient does not exist in the medical research fraud detection system and does the operator want to proceed to registering the new patient. In some embodiments, if there was no patient parameter match, the fraud detection application software may determine if the patient is a high-risk patient 484 and deny enrollment if the patient is determined to be a high-risk patient. In some embodiments, if the fraud detection application software determines the patient is not a high-risk patient, the fraud detection application software may move forward 485 with the registration process.

In some embodiments, if there is a patient parameter match, the patient exists at another site, the patient is not currently not undergoing any trial or study, but has not passed the predetermined time window, the patient may be declined enrollment in the current study or trial 486. In some embodiments, a message may be generated identifying “This Patient has not met 30 days window period between clinical trials. Patient eligible to enroll on [Exact Date]”.

In some embodiments, if there is a patient parameter match, the patient exists at the other site or the current site, and the patient is currently undergoing a trial at the other or another Site, the fraud detection software may decline to enroll the patient in the current study or trial 487. In some embodiments, the fraud detection software may communicate a warning message, such as “The patient exists, and is currently enrolled in a clinical trial at another Site.” In some embodiments, the message may be communicated in a bright or identifying color (e.g., red color).

In some embodiments, if there is a patient parameter match, the patient exists at the current site and is currently undergoing the trial at the current site, the fraud detection software may decline to enroll the patient in the current study or trial 487. In some embodiments, the fraud detection software application may communicate a warning message, “The patient exists, and is currently enrolled in a clinical trial at my Site.” In some embodiments, the message may be communicated in a bright or loud color (e.g., red color) to deny the enrollment.

In some embodiments, if there is a patient parameter match, the patient exists at another site, or the current site, is not currently enrolled in a current trial, and has passed a predetermined time window, the patient may be eligible to be enrolled 488 in the current study or trial. In some embodiments, a message may be generated identifying “This Patient is present at some other Site or at this site, is currently not undergoing any trial, and has crossed 30 days window period after the Trial. Do you want to pull this patient to your Site, and add in the trial.”

In some embodiments, if the patient is eligible to be enrolled in the trial or study, the fraud detection software may evaluate the patient's record in the database to determine whether the patient is a high-risk patient. If the patient is determined to be a high-risk patient, the fraud detection software may deny the patient enrollment in the current trial or study 489. In some embodiments, an administrator or sponsor may override the denial of enrollment by the fraud detection software if they determine the high-risk patient may be in the current trial.

The ability to designate patients as high-risk patients for clinical studies based on previous actions is another unique aspect to the clinical trial fraud detection software application. There is no system that is able to obtain this information from the many sources (or computing devices) to determine whether or not a patient is a high risk to leave the clinical trial or study due to bad behavior or prior actions. In some embodiments, the medical research fraud detection software includes this feature for analyzing a patient's record and/or other records in the medical research fraud detection system in order to identify whether high-risk behavior has been exhibited in the past. In some embodiments, the fraud detection software application may analyze the electronic history of the patient's behavior in previous studies in order to determine if the patient is high-risk. In some embodiments, this determination is based on actions of patient as recorded in the patient records and/or clinical trial records, and not on medical history of the patient (in other words, they are not analyzing the patient's medical response to the trial; instead the system is focused on whether the patient followed protocol, completed the study, and/or exhibited professional behavior during the clinical study.

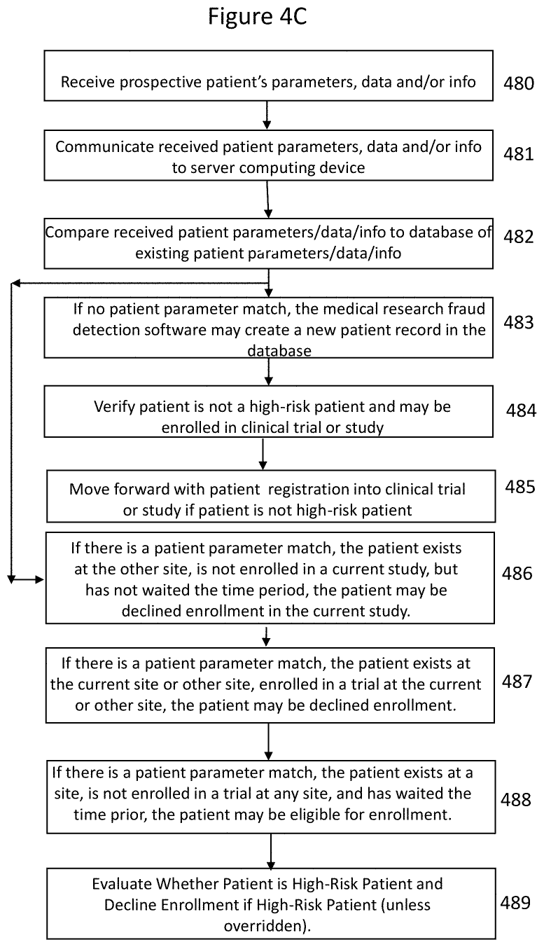

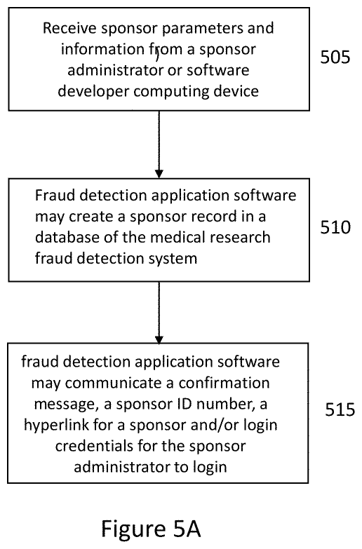

FIG. 5A illustrates a process of sponsor activation in the medical clinical trial fraud detection software system according to some embodiments. In some embodiments, the fraud detection application software may receive sponsor parameters and information 505 from a sponsor administrator or software developer computing device. In some embodiments, the sponsor parameters or information may include company name, abbreviation, tax ID number, office phone number, fax number, mailing address, account administrator name, account administrator email ID, and/or account administrator phone contacts. In some embodiments, the fraud detection application software may create 510 a sponsor record in a database of the medical research fraud detection system. In some embodiments, the database may be a sponsor database or a combined database including one or more of the other databases in the medical research fraud detection system. In some embodiments, the fraud detection application software may communicate 515 a confirmation message, a sponsor ID number, a hyperlink for a sponsor and/or login credentials for the sponsor administrator to login to the fraud detection application software. In some embodiments, the confirmation message may be communicated to the sponsor administrator computing device (or the software developers or administrator's device).

FIG. 5B illustrates a process of user authentication in a medical research fraud detection system according to some embodiments. In some embodiments, all users of the medical clinical trial fraud detection software may be able to establish login credentials. In some embodiments, the users may be an IDC administrator, sponsor administrator, CRO Principal Investigator, an Investigator and/or a study coordinator. Each of these different users may be able to establish different roles and/or responsibilities as well as access in the medical clinical fraud detection system. In some embodiments, the user may first need to create the user credentials. In some embodiments, upon user creation of the credentials, the fraud detection software application may receive 525 the user credentials (including but not limited to, the username, the password, a language preference and an indicator that the registration hyperlink has been pressed) from the user computing device. In some embodiments, the fraud detection software application may verify 530 the user does not have an account and may create an account for the user. In some embodiments, the fraud detection software application may communicate 535 to the user computing device that the user account has been created. After creation of the account and during a normal login to the fraud detection system, in some embodiments, the fraud detection software application may receive 540 the user credentials. In some embodiments, the fraud detection software application may authenticate 545 the user credentials and may re-direct the user to the respective web page (e.g., CRO page, sponsor page, IDC administrator page, etc.). In some embodiments, the fraud detection software application may also include forget password functionality and/or change password functionality. In some embodiments, the fraud detection software application may also include functionality to view or edit user profiles, parameters or information. In some embodiments, the fraud detection software application may also include functionality to view or edit sponsor profiles, parameters or information.

In some embodiments, the fraud detection software application may allow a sponsor administrator to create, edit and/or update payment details for the payments that the sponsor financial institution pays to the IDC (or software developer) administrator. In some embodiments, the fraud detection software application may receive e-check details or parameters, ACH details or parameters, credit card details and/or parameters, and/or debit card details and/or parameters from the sponsor administrator computing device.

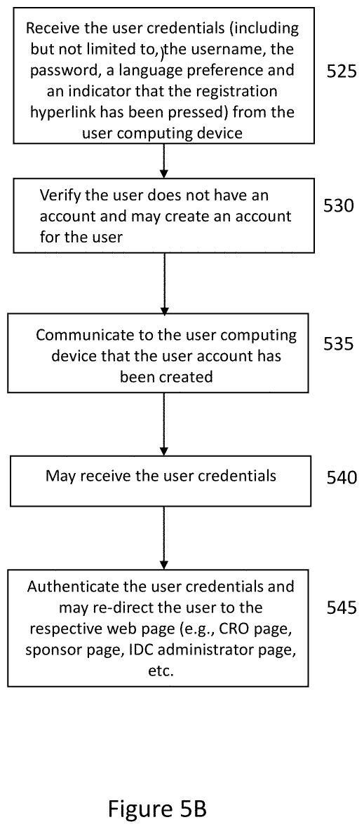

FIG. 6 illustrates a process for initiating or creating a clinical study or clinical trial in the medical research fraud detection system according to some embodiments. In some embodiments, the fraud detection software application may allow creation of a clinical study or clinical trial by a sponsor. In some embodiments, a sponsor administration may be able to enter the study details and/or parameters. In some embodiments, the fraud detection software application may receive the study details and/or parameters 605 from the sponsor administrator computing device. In some embodiments, the study details and/or parameters may include a study name, a study phase, a NCT number, a protocol number, a study description, a study medical description, a study age group, a study gender, a study race/ethnicity, a study registration fee, and/or a study subscription plan. In some embodiments, the fraud detection software application may receive any exclusion parameters relating to the created study 610 from the sponsor administrator computing device. In some embodiments, exclusion parameters may include how many previous studies the sponsor may allow patients to have participated in. In some embodiments, the fraud detection software application may verify the NCT number is authentic 615. In some embodiments, the fraud detection software application may communicate with a third-party or external computing device that has a database of valid NCT numbers. In some embodiments, the fraud detection software application may internally check a database in the medical research fraud detection system to verify the received NCT number is valid. In some embodiments, the fraud detection software application may create 620 a clinical study record for the new study which may be stored in a database. In some embodiments, the database may be a clinical study database or may be a combined database that also includes patient records, sponsor records, and/or clinical study records. In some embodiments, the fraud detection software application may receive a payment confirmation 625 from a third-party processor computing device or from the sponsor computing device themselves and the new study may then be registered in the medical research fraud detection system. In some embodiments, the fraud detection software application may allow a sponsor administrator to format or configure a format of a patient's study ID. In some embodiments, the fraud detection software application may receive 630 patient study ID format instructions from the sponsor computing device and thus verify that any patient study IDs follow this format and/or configure the patient study IDs to follow this format. In some embodiments, the patient study ID may be a combination of a prefix value and/or a suffix value. In some embodiments, the patient ID prefix value may be a combination of sponsor ID, study ID and/or site ID. In some embodiments, the patient ID suffix value may be a unique ID generated by the fraud detection software application at run-time and may be related to the type and size of patient ID suffix value. In some embodiments, the fraud detection software application may allow a sponsor administrator to close or mark as completed a clinical trial or study. This may occur at one site and/or different sites. In some embodiments, the fraud detection software application may receive 635 clinical trial study completion parameters or information and complete the setup of the clinical study.

In some embodiments, the fraud detection software application may allow a sponsor administrator to search for clinical studies and/or also view details of clinical studies. In some embodiments, the clinical studies may be searched by Study ID, Study Name, Study Phase, NCT Number and/or Protocol Number. In some embodiments, the details and/or parameters of clinical studies may be viewed with the above-identified parameters with up to 15 clinical studies listed on a page. In some embodiments, the fraud detection software application may allow editing of the study details and/or parameters of clinical studies. In some embodiments, the fraud detection software application may allow viewing of the payment summary of each of the clinical trials or studies. In some embodiments, for example, the fraud detection software application may allow viewing of the payment details such as study registration fee, plan type, study monthly fee, study subject exclusion fee, study total fee, payment type, date of payment, and/or transaction ID for each of the clinical trials and/or studies.

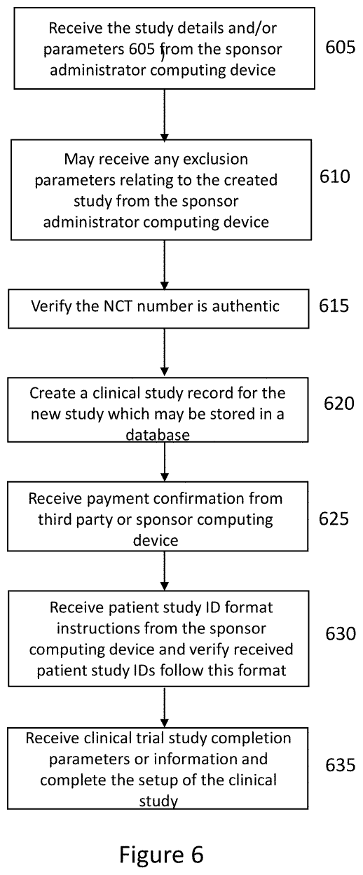

FIG. 7 illustrates a clinical trial or study site management process according to some embodiments. In some embodiments, the fraud detection software may allow a sponsor administrator to create a clinical study site record for each clinical study site. In some embodiments, the fraud detection software may receive clinical study site parameters or information 705 for a new clinical site. In some embodiments, the clinical study site parameters or information may include a site name, a study name, an address, a geographic area, a phone number and/or a fax number In some embodiments, the clinical study site parameters or information may include principal investigator parameters or details, such as name, email address or email ID, and/or phone contact information. In some embodiments, the clinical study site parameters or information may include study coordinator parameters or details, such as name, email address or email ID, and/or phone contact information. In some embodiments, the fraud detection software may create a clinical site record for the new clinical site and/or generate a site ID for the new clinical site 710. In some embodiments, the clinical site record may be stored in a database. In some embodiments, the database may be a clinical site database or a clinical trial database. In some embodiments, the database may be a combined database including all or some of the previously configured databases. In some embodiments, the fraud detection software may communicate a confirmation message 715 to the sponsor administrator computing device to confirm that the new clinical site record has been created and the site ID has been created for the clinical site. In some embodiments, the fraud detection application software may allow activation of the new clinical site. In some embodiments, the fraud detection application software may receive an activation parameter or indicator 720 identifying that the new clinical site has been activated and may communicate that the site has been activated to the sponsor administrator computing device. In some embodiments, the fraud detection application software may perform the record creation for a plurality of additional clinical sites. In some embodiments, the fraud detection application software may include functionality to allow searching and/or viewing of different clinical trial sites and/or associated parameters or information by, for example, a sponsor administrator. In some embodiments, the fraud detection application software may include functionality to allow for editing of different clinical trial site records and/or associated parameters or information. In some embodiments, the fraud detection application software may include functionality to allow for deactivation of different clinical trial sites and/or different clinical trial site records.

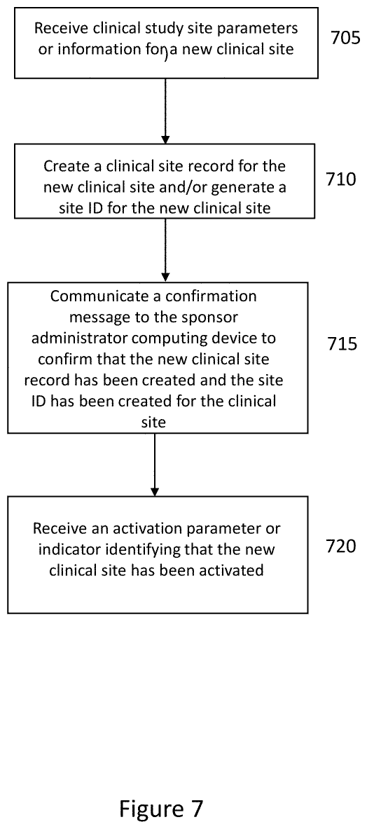

FIG. 8 illustrates a clinical study site assignment process according to some embodiments. In some embodiments, the fraud detection application software may allow a sponsor administrator to assign clinical studies or trials and/or respective sites to a clinical research organizer (CRO). In some embodiments, the fraud detection application software may receive clinical study assignment parameters or information and/or associated site parameters 805 for a sponsor administration computing device. In some embodiments, the clinical study assignment parameters or information and/or associated site parameters may include one or more selected clinical studies (and assigned CRO), one or more selected clinical sites associated with the selected clinical studies (and assigned CRO), one or more assignment date values and/or related comments. In some embodiments, based on the received clinical study assign parameters or information and/or associated site parameters, the fraud detection application software may assign 810 the CRO to the one or more selected clinical studies and/or the associated one or more clinical sites. In some embodiments, the fraud detection application software may verify that the CRO can be and has been assigned 815 to the one or more selected clinical studies and/or the one or more associated clinical sites. In some embodiments, the fraud detection application software may communicate 820 a message to the sponsor (or sponsor computing device) and/or the clinical research organizer (CRO) or CRO computing device verifying that the study and/or site has been assigned. In some embodiments, the fraud detection application software may allow a sponsor administrator to view assigned study(s) and/or site(s) for one or more CROs (or CRO computing device). In some embodiments, the fraud detection application software may allow a sponsor administrator to edit assigned study(s) and/or site(s) for one or more CROs (or CRO computing device). In some embodiments, the fraud detection application software may allow a sponsor administrator to unassign clinical study(s) and/or site(s) for one or more CROs.

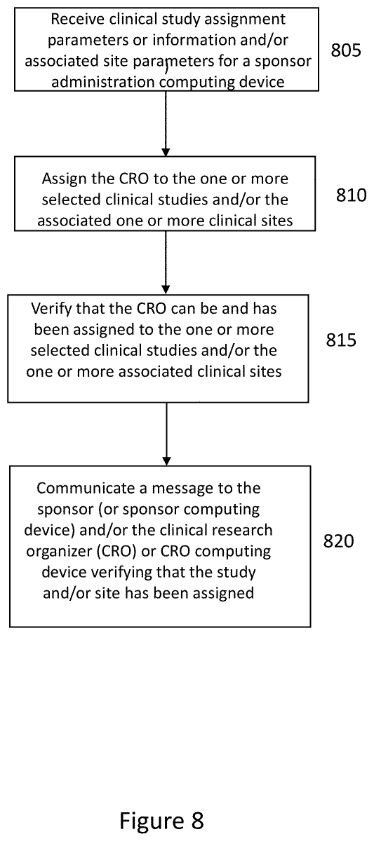

FIG. 9 illustrates a medical research fraud detection system financial management process according to some embodiments. In some embodiments, a software developer (e.g., BiolDC) administrator may be able to configure payment or financial parameters for clinical studies. In some embodiments, the fraud detection software application may receive 905 clinical study financial parameters for clinical study(s) or trial(s). In some embodiments, the financial parameters may include a study registration fee, a subject exclusion fee, a subscription plan information or fees, and/or subject verification fees. In some embodiments, the fraud detection software application may publish or communicate 910 the financial parameters to all clinical sponsors (and/or clinical sponsor computing device). In some embodiments, the fraud detection software application may allow software developer administrators to view financial parameters for clinical studies. In some embodiments, the fraud detection software application may allow software developer administrators to edit the financial parameters for clinical studies. In some embodiments, the fraud detection software application may allow software developer administrators to view payment histories for subscription plans for clinical sponsor's studies. In some embodiments, the fraud detection software application may allow software developer administrators to view payment histories or financial parameters for study registration fees and/or subject exclusion fees for clinical sponsor's studies.

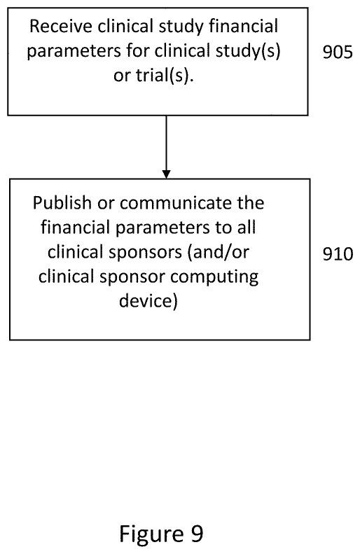

FIG. 10 illustrates an auditing process in the medical research fraud detection system according to some embodiments. In some embodiments, the medical research fraud detection software may generate audit trails for different activities within the medical research fraud detection system. In some embodiments, in step 1010, the medical research fraud detection software application may generate audit logs at a software developer level when specific conditions or activities occur. In some embodiments, the activities and/or conditions when the fraud detection software log generates audit trails or logs at a software developer level (IDC level) includes: 1) a new sponsor is registered or its profile is updated; 2) any users are created, edited, deactivated and activated; 3) a patient is deactivate; 4) a sponsor account is frozen; 5) a sponsor account is activated; 6) a user logs in or logs out as well as when login attempts are made. In some embodiments, in step 1020, the medical research fraud detection software application may generate audit logs at a sponsor or sponsor administrator level when specific conditions or activities occur. In some embodiments, the activities and/or conditions when the fraud detection software log generates audit trails or logs at a sponsor or sponsor administrator level includes: 1) payments are registered via eCheck, ACH or credit/debit cards; 2) user profiles; company profiles, and payment details are updated; 3) a new study is created and its details are updated; 4) payments are completed for a study signup; 5) monthly payments are made; 6) a patient's study ID is configured; 7) a new site is added and/or its details are updated; 8) any user is created, edited, deactivated or activate at a sponsor level; 9) studies are assigned and the study sites are assigned to a CRO; 10) payment is registered at a site; 11) any patient information or parameters is updated; 12) a QR code is generated; and 13) a clinical trial is started where a patient is enrolled in a trial; 14) a patient is exited from a trial; 15) a patient is marked as high-risk; 16) white labeling configuration settings applied; 17) a new master role is added, or an existing role is deleted, edited at a sponsor level; and 18) sponsor users login, logout, or have failed login attempts. In some embodiments, in step 1030, the medical research fraud detection software application may also generate audit logs whenever there are any exceptions or errors that occur in the fraud detection application software. In some embodiments, in step 1040, the medical research fraud detection software application may also generate audit logs with respect to any database activities, such as create record(s), edit record(s), and/or delete record(s). In some embodiments, the fraud detection software may also capture the following information: type of information, description, date, time stamp, Sponsor ID, Sponsor Name, Study ID, Study name, by whom, user role, table name, column name, record ID, original data and/or new data. In some embodiments, an Integrated Data Check administrator (on their computing device) may be able to view audit logs generated by the fraud detection software application. In some embodiments, the sponsor administrator (on their computing device) may be able to view audit logs generated by fraud detection software application. In some embodiments, the medical research fraud detection software may also secure patient data in order to ensure HIPAA compliance. In some embodiments, the medical research fraud detection software may verify that only authorized users may access patient data in the patient database; that any patient data should be encrypted in-transit and/or at rest; that patient identifiers such as SSN, ID/DL number should be encrypted and that no patient ID should be exposed in any URL request.

In some embodiments, the medical research fraud detection software may allow a sponsor administrator (on their computing device) to create, view, and/or edit the CRO, the investigator and/or the study coordinator users in the fraud detection software. In some embodiments, when creating the user, the medical research fraud detection software may receive first name, last name, role (e.g., CRO, investigator, study coordinator), study name, site name, email address or ID, mobile phone number or office phone number. In some embodiments, the user data may be stored in a user database, or in a combined database in the medical research fraud detection system. In some embodiments, the sponsor administrator (through their computing device) may be able to search users, access the list or grid of users, activate and/or deactivate the users in the fraud detection software application.

In some embodiments, the medical research fraud detection software application may allow a sponsor administrator (through a computing device) to add, edit and/or delete the user records (e.g., the user roles) of sponsor employees in the medical research fraud detection system. In some embodiments, the system administrator (through their computing device) may be able add, edit and/or delete roles in the fraud detection software application. In some embodiments, the system administrator (through their computing device) may configure the privileges and/or access rights of the features and/or screens (against the user's roles) in the fraud detection software application. In some embodiments, this means identifying whether certain users can read, write or delete data and/or parameters in certain features and/or screens. In some embodiments, the features for which the privileges and access rights may be edited, added or deleted may be the sponsor company profiles, monthly subscription payment, registration payment details, management of clinical studies, management of sites, management of trials, management of patients, the dashboard, user management at a sponsor level, white labeling, notifications and/or payments for account activation.

In some embodiments, the medical research fraud detection software application may allow a software administrator (e.g., Integrated Data Check administrator) to create, view, edit, deactivate and/or activate other software administrator (e.g., IDC administrators) in the medical research fraud detection system and software. In some embodiments, the software administrator users may include the following information in the user record: name, address, role, email address or ID, mobile phone, and/or office phone. In some embodiments, the user record may be stored in a user database and/or a combined database. In some embodiments, the software administrator (though their computing device) may search and/or list other software administrator users in the fraud detection software. In some embodiments, the software administrator (through their computing device) may view details of other software administrator users, edit details of other software administrator users, activate software administrator users and/or deactivate software administrator users and have this information saved in the user's records.

In some embodiments, the medical research fraud detection software may allow a software administrator (e.g., a Integrated Data Check (IDC) administrator) to search the sponsors, access the sponsors list and/or grid, view the sponsor details, deactivate sponsors, activate sponsors, activate and/or delete sponsors in sponsor records in a sponsor database (or a combined database) in the medical research fraud detection system and/or software application. In some embodiments, the medical research fraud detection software application may allow a software administrator (e.g., IDC administrator) through their computing device to search studies, access the study list or grid, view study details, and/or delete the study details in study records in a study database (or a combined database) in the medical research fraud detection system and/or software application. In some embodiments, the medical research fraud detection software application may allow a software administrator (e.g., IDC administrator) to search clinical sites, access the clinical sites list/grid, view the clinical site details and/or delete the site in the site records in a site database and/or a combined database in the medical research fraud detection system and/or software application. In some embodiments, the medical research fraud detection software application may allow a software administrator (e.g., IDC administrator) to manage patients by searching the patient records, accessing the patient record list/grid, view the patient record details, and/or delete patient records in a patient database (and/or a combined database) in the medical research fraud detection system and/or software application. In some embodiments, the medical research fraud detection software application may allow a software administrator (e.g., IDC administrator) to manage users may searching user records (e.g., CRO, principal investigator records, investigator records, and study coordinator records), accessing the users list/grid, view the user records and/or delete the users in the user records of a user database and/or a combined database in the medical research fraud detection system and/or software application. In some embodiments, the medical research fraud detection software application may allow a software administrator (e.g., IDC administrator) to view a top fire most recent notifications and/or all notifications in the in the medical research fraud detection system and/or software application.

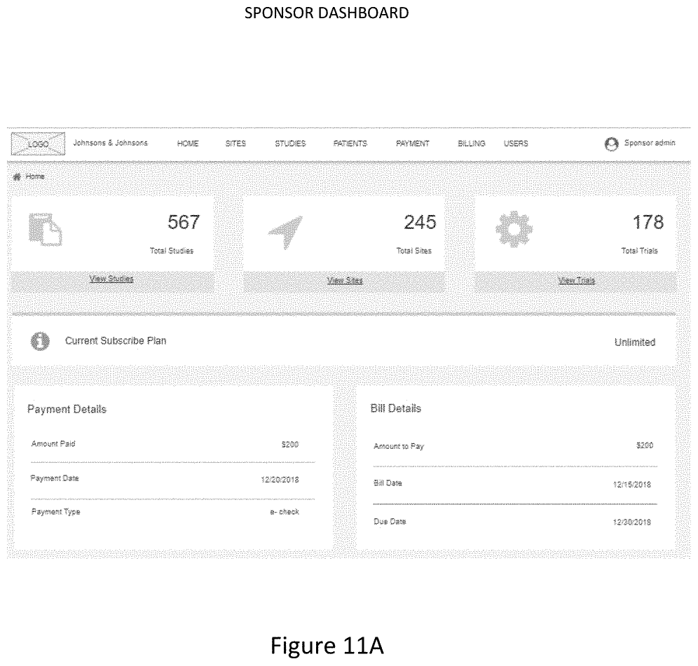

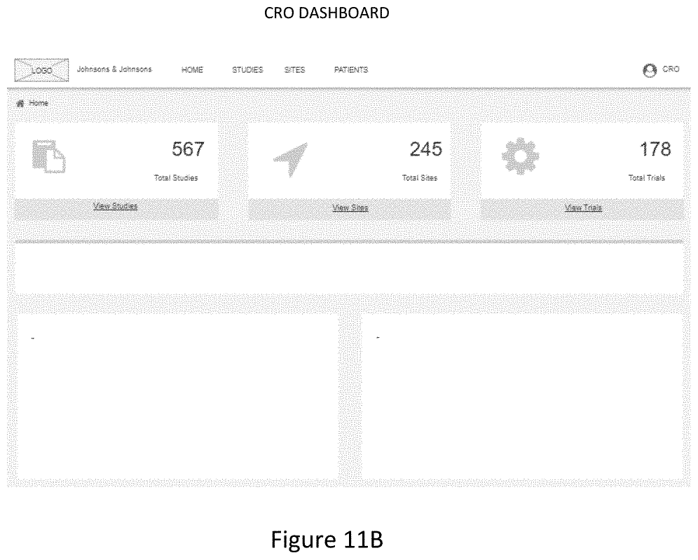

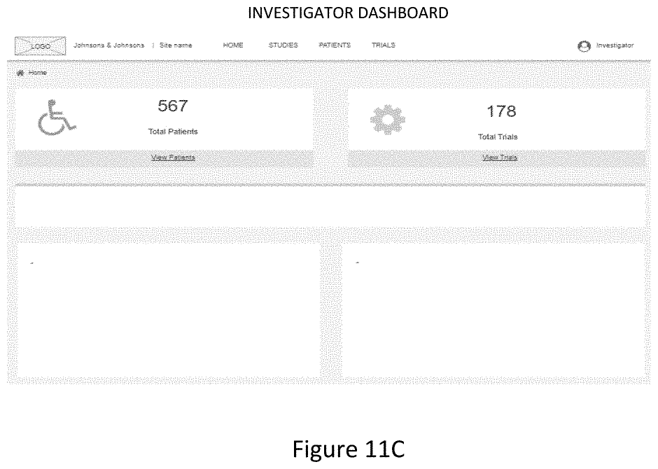

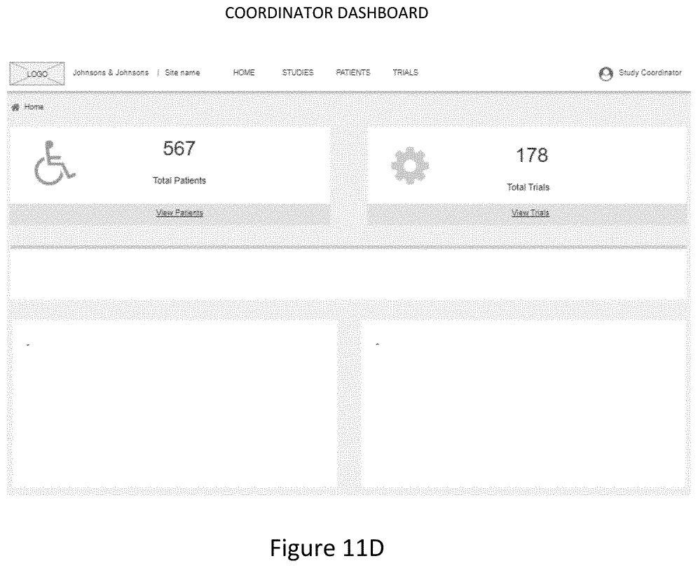

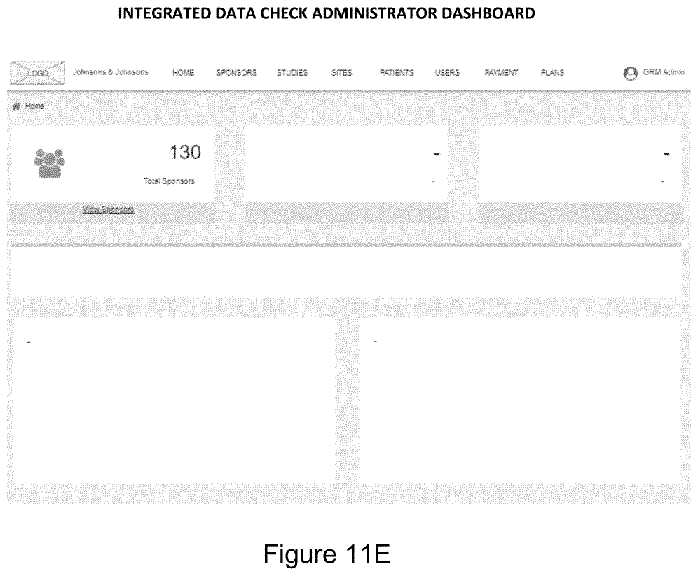

FIGS. 11A-11F illustrates dashboards or user interface screens in the medical research fraud detection software application according to some embodiments. FIG. 11A illustrates a sponsor administrator dashboard in a medical research fraud detection software application according to some embodiments. In some embodiments, the medical research fraud detection software application may generate a sponsor administrator dashboard (or user interface screen) after a sponsor administrator (through their computing device) enters their login information. In some embodiments, the fraud detection software application may generate the sponsor administrator dashboard which may include 1) a total studies label with a counter and a view studies hyperlink (to a manage studies screen); 2) a total sites label with counters and a view sites hyperlink (to a manage sites screen); 3) a total trials label with counters and a view trials hyperlink (to a manage patient trials screen); 4) a current subscribe plan label; 5) a payment details section or menu with amount paid, amount due and payment amount details; and/or 6) a bill details section.

FIG. 11B illustrates a CRO dashboard in a medical research fraud detection software application according to some embodiments. In some embodiments, the medical research fraud detection software application may generate a CRO dashboard or user interface screen after a CRO enters their login information. In some embodiments, the fraud detection software application may generate the CRO dashboard which may include a 1) a total studies label with a counter and a view studies hyperlink (to a manage studies screen); 2) a total sites label with counters and a view sites hyperlink (to a manage sites screen); and/or 3) a total trials label with counters and a view trials hyperlink (to a manage patient trials screen).

FIG. 11C illustrates an investigator dashboard in a medical research fraud detection software application according to some embodiments. In some embodiments, the medical research fraud detection software application may generate an investigator dashboard (or user interface screen) after the investigator enters in the login information. In some embodiments, the fraud detection software application may generate the investigator dashboard which may include a total patients label with counters, and a view patients hyperlink (to a manage patients screen or menu) and a total trials label with counters with a view trials hyperlink (to a manage patient trials screen or menu).

FIG. 11D illustrates a study coordinator dashboard in a medical research fraud detection software application according to some embodiments. In some embodiments, the medical research fraud detection software application may generate a study coordinator dashboard (or user interface screen) after the study coordinator enters in the login information. In some embodiments, the fraud detection software application may generate the study coordinator dashboard which may include a total patients label with counters, and a view patients hyperlink (to a manage patients screen or menu) and a total trials label with counters with a view trials hyperlink (to a manage patient trials screen or menu).

FIG. 11E illustrates a software administrator (e.g., IDC administrator) dashboard in a medical research fraud detection software application according to some embodiments. In some embodiments, the medical research fraud detection software application may generate a software administrator (e.g., IDC administrator) dashboard or user interface screen) after the software administrator enters their login information. In some embodiments, the fraud detection software application may generate the software administrator dashboard which may include a sponsors label with counters and a view sponsors hyperlink (which may be redirected to a manage sponsors screen or menu).

In some embodiments, a method of detecting fraudulent patient behavior in a clinical research study includes accessing computer-readable instructions from one or more physical memory devices for execution by one or more processors; executing the computer-readable instructions accessed from the one or more physical memory devices by the one or more processors; storing, in at least one of the physical memory devices, values resulting from having executed the computer-readable instructions on the one or more processors; and wherein the accessed computer-readable instructions to detect fraudulent patient behavior. The method includes executing fraudulent patient behavior instructions including: receiving, at a computing device, a patient fingerprint image, from an imaging device of administrator or investigator computing device; comparing, at the computing device, the received patient fingerprint image to a database of existing patient fingerprint images to determine if the patient is previously enrolled in a clinical study; communicating a message identifying the patient may be eligible to enroll if no match is found between the received fingerprint image and the database of existing patient fingerprint images; and performing additional analysis with respect to the patient if a match is found between the received fingerprint image and the database of existing fingerprint images. In some embodiments, if no match is found between the received fingerprint image and the database of existing fingerprint images, then executing the fraudulent patient behavior instructions further includes analyzing an existing patient electronic record to determine whether the patient is a high-risk patient; and initially denying enrollment into the clinical study if the patient is a high-risk patient. In some embodiments, executing the fraudulent patient behavior instructions further includes: overriding, by an external computing device, the initial denial of enrollment by a sponsor administrator; and communicating an electronic message indicating acceptance of patient's enrollment into the clinical study. In some embodiments, executing the fraudulent patient behavior instructions further includes receiving a patient voice file and storing the patient voice file in the patient record. IN some embodiments, executing the fraudulent patient behavior instructions further includes receiving a consent document image electronically signed and storing the consent document image in the patient record. In some embodiments, executing the fraudulent patient behavior instructions further includes: receiving patient identification parameters or information; generating a QR code, the QR code representative of the received patient identification parameters or information; and storing the QR code in the patient record.

In some embodiments, the performing the additional analysis with respect to the patient if a match is found between the received fingerprint image and the database of existing fingerprint images, further includes analyzing whether the patient has waited a predetermined time window after a prior clinical study; and if the patient has not waited the predetermined time window, communicating a message to the sponsor or administrator computing device denying the patient enrollment into the clinical study. In some embodiments, the performing the additional analysis with respect to the patient if a match is found between the received fingerprint image and the database of existing fingerprint images, further includes analyzing whether the patient has waited a predetermined time window after a prior clinical study, if the patient has waited the predetermined time window, analyzing whether the patient is currently enrolled in another clinical study; and if the patient is currently enrolled in another clinical study, communicating a message to the administrator computing device denying the patient enrollment into the clinical study. In some embodiments, executing the fraudulent patient behavior instructions further includes analyzing the patient record to determine whether the patient has been lost to follow-up for more than a set number of clinical studies during a pre-determined timeframe; and communicating a message to the patient initially denying enrollment in the clinical study if the patient has been lost to follow-up for more than the set number of clinical studies during the predetermined timeframe. In some embodiments, executing the fraudulent patient behavior instructions further comprising: analyzing the patient record to determine whether the patient has withdrawn consent more than a set number of clinical studies during a predetermined timeframe; and communicating a message to the patient initially denying enrollment in the clinical study if the patient has withdrawn consent for more than the set number of clinical studies during the predetermined timeframe. In some embodiments, executing the fraudulent patient behavior instructions further includes analyzing the patient record to determine if the patient has been withdrawn by an investigator, a sponsor administrator or a software developer; and communicating a message to the patient initially denying enrollment in the clinical study if the patient has been withdrawn by the investigator, the sponsor administrator or the software developer. In some embodiments, executing the fraudulent patient behavior instructions further includes analyzing the patient record to determine if the patient has had more than three screen failures in the last year; and communicating a message to the patient initially denying enrollment in the clinical trial if the patient has had more than three screen failures in the last year.

In some embodiments, a system to detect patient fraud in clinical trials or studies includes one or more memory devices, the one or more memory devices including a database to store a plurality of patient records; one or more processors; and computer-readable instructions, the computer-readable instructions stored in the one or more memory devices in a different storage area from the database. In some embodiments, the computer-readable instructions are executable by the one or more processors to: receive clinical study details or parameters for a clinical study; receive a clinical study start date, a clinical study end date and clinical study comments, all for the clinical study; create a clinical study record, the clinical study record to include the clinical study details or parameters and store the clinical study record in the database; receive a request to add a patient to the clinical study; verify the patient is enrollment eligible for the clinical study utilizing fingerprint validation, the fingerprint validation including comparing a received image of the patient's fingerprint to a plurality of existing patient fingerprint images in the database; and create a patient record, the patient record to be associated with the clinical study. In some embodiments, the computer-readable instructions are further executable by the one or more processors to: if the patient is verified to be enrollment eligible, verify that the patient record does not include a high-risk indicator and allow the patient to move towards registration if the patient record does not include the high-risk indicator. In some embodiments, the computer-readable instructions are further executable by the one or more processors to: if the patient is verified to be enrollment eligible, further verity that the patient record does not include a high-risk indicator and if the patient record includes the high risk indicator, allowing the patient to move towards registration if an overriding authorization indicator or parameter is received from a sponsor administrator computing device. In some embodiments, the computer-readable instructions are further executable by the one or more processors to proceed with registration and receive registration parameters from the eligible patient. In some embodiments, the computer-readable instructions are further executable by the one or more processors to receive a patient's voice file and to store the patient's voice file in the database. In some embodiments, the computer-readable instructions are further executable by the one or more processors to: receive a patient's electronically signed consent form image; and store the patient's electronically signed consent form image in the database. In some embodiments, the computer-readable instructions further executable by the one or more processors to generate a QR code for the enrolled patient, the QR code including representations of a patient's parameters; and to store the QR code in the database. In some embodiments, a method of detecting fraudulent patient behavior in a clinical research study includes accessing computer-readable instructions from one or more physical memory devices for execution by one or more processors; executing the computer-readable instructions accessed from the one or more physical memory devices by the one or more processors; storing, in at least one of the physical memory devices, values resulting from having executed the computer-readable instructions on the one or more processors, wherein the accessed computer-readable instructions to detect fraudulent patient behavior. In some embodiments, executing fraudulent patient behavior instructions includes receiving, at a computing device, patient parameters, from an administrator or investigator computing device; comparing, at the computing device, the received patient parameters to a database of existing patient parameters to determine if the patient is previously enrolled in a clinical study; communicating a message identifying the patient may be eligible to enroll if no match is found between the received patient parameters and the database of existing patient parameters; and performing additional analysis with respect to the patient if a match is found between the received patient parameters and the database of existing fingerprint parameters.

In some embodiments, computing device may communicate with each other in order to engage in the medical research fraud detection system For example, first and third computing devices may be capable of rendering a GUI via a device, such as a network device and/or a computing device, for example, so that a user-operator may engage in operations of the medical research fraud detection system. Likewise, third computing device may serve a similar function as computing second device in this example. In embodiments, first computing device may interface with second computing device, which may comprise features, for example, including communications interface, one or more processors (e.g., processing units), one or more memory devices, which may comprise one or more primary memory devices and secondary memory devices, may communicate by way of a communication bus, for example. In some embodiments, a first computing device may represent one or more sources of medical research fraud detection instructions in the form physical states and/or signals, for example. In some embodiments, the first computing device may communicate with the second computing device by way of a network connection. Although the second computing device includes the above-identified components, claimed subject matter is not limited to computing devices having only these components as other implementations may include alternative arrangements that may comprise additional components or fewer components, such as components that function differently while achieving similar results. Rather, examples are provided merely as illustrations. It is not intended that claimed subject matter to limited in scope to illustrative examples.

In some embodiments, the one or more processors may be representative of one or more circuits, such as digital circuits, to perform at least a portion of a computing procedure and/or process. By way of example, but not limitation, the one or more processors may include controllers, microprocessors, microcontrollers, application specific integrated circuits, digital signal processors, programmable logic devices, field programmable gate arrays, the like, or any combination thereof. In implementations, the one or more processors may perform signal processing to manipulate signals and/or states, to construct signals and/or states, etc., for example.

In some embodiments, the one or more memory devices may be representative of any storage mechanism. In some embodiments, the one or more memory devices may comprise, for example, primary memory devices and secondary memory devices, additional memory circuits, mechanisms, or combinations thereof may be used. In some embodiments, the one or more memory devices may comprise, for example, random access memory, read only memory, etc., such as in the form of one or more storage devices and/or systems, such as, for example, a disk drive, an optical disc drive, a tape drive, a solid-state memory drive, etc., just to name a few examples. Inn some embodiments, the one or more memory devices may be utilized to store a software application program or computer-readable instructions. In some embodiments, the one or more memory devices may also comprise a memory controller for accessing computer readable-medium that may carry and/or make accessible content, which may include code, and/or instructions, for example, executable by the one or more processors and/or some other unit, such as a controller and/or processor, capable of executing instructions, for example.

In some embodiments, the network may comprise one or more network communication links, processes, services, applications and/or resources to support exchanging communication signals between a first computing device, such as second computing device, and third (or second) computing device 6. By way of example, but not limitation, network may comprise wireless and/or wired communication links, telephone and/or telecommunications systems, such as a local area network (LAN).

The term “computing device,” as used herein, refers to a system and/or a device, such as a computing apparatus, that includes a capability to process (e.g., perform computations) and/or store content, such as measurements, text, images, video, audio, etc. in the form of signals and/or states. Thus, a computing device, in this context, may comprise hardware, software, firmware, or any combination thereof (other than software per se). Computing device is merely one example, and claimed subject matter is not limited in scope to this particular example. For one or more embodiments, a computing device may comprise any of a wide range of digital electronic devices, including, but not limited to, personal desktop and/or notebook computers. Further, unless specifically stated otherwise, a process as described herein, with reference to flow diagrams and/or otherwise, may also be executed and/or affected, in whole or in part, by a computing platform.

Regarding aspects related to a communications and/or computing network, a wireless network may couple other devices with a network. A wireless network may include virtually any type of now known and/or to be developed wireless communication mechanism by which signals may be communicated between devices, between networks, within a network, and/or the like. Communications between a computing device and/or a network device and a wireless network may be in accordance with known and/or to be developed communication network protocols.

A device, such as a computing and/or networking device, may vary in terms of capabilities and/or features. Claimed subject matter is intended to cover a wide range of potential variations. A computing and/or network device may include and/or may execute a variety of now known and/or to be developed operating systems, derivatives and/or versions thereof, including personal computer operating systems, such as a Windows, iOS, Linux, a mobile operating system, such as iOS, Android, Windows Mobile, and/or the like. A computing device and/or network device may include and/or may execute a variety of possible applications, such as a client software application enabling communication with other devices. A network may communicate via signal packets and/or frames, such as in a network of participating digital communications. The foregoing is provided merely to illustrate that claimed subject matter is intended to include a wide range of possible features and/or capabilities.

Algorithmic descriptions and/or symbolic representations are examples of techniques used by those of ordinary skill in the signal processing and/or related arts to convey the substance of their work to others skilled in the art. An algorithm is here, and generally, is considered to be a self-consistent sequence of operations and/or similar signal processing leading to a desired result. In this context, operations and/or processing involves physical manipulation of physical quantities. Typically, although not necessarily, such quantities may take the form of electrical and/or magnetic signals and/or states capable of being stored, transferred, combined, compared, processed or otherwise manipulated as electronic signals and/or states representing various forms of content, such as signal measurements, text, images, video, audio, etc. It has proven convenient at times, principally for reasons of common usage, to refer to such physical signals and/or physical states as bits, values, elements, symbols, characters, terms, numbers, numerals, measurements, content and/or the like. It should be understood, however, that all of these and/or similar terms are to be associated with appropriate physical quantities and are merely convenient labels. Unless specifically stated otherwise, as apparent from the preceding discussion, it is appreciated that throughout this specification discussions utilizing terms such as “processing,” “computing,” “calculating,” “determining”, “establishing”, “obtaining”, “identifying”, “selecting”, “generating”, and/or the like may refer to actions and/or processes of a specific apparatus, such as a special purpose computer and/or a similar special purpose computing and/or network device. In the context of this specification, therefore, a special purpose computer and/or a similar special purpose computing and/or network device is capable of processing, manipulating and/or transforming signals and/or states, typically represented as physical electronic and/or magnetic quantities within memories, registers, and/or other storage devices, transmission devices, and/or display devices of the special purpose computer and/or similar special purpose computing and/or network device. In the context of this particular patent application, as mentioned, the term “specific apparatus” may include a general purpose computing and/or network device, such as a general purpose computer, once it is programmed to perform particular functions pursuant to instructions from program software.

The above disclosure is sufficient to enable one of ordinary skill in the art to practice the invention, and provides the best mode of practicing the invention presently contemplated by the inventor. While there is provided herein a full and complete disclosure of the preferred configurations of this invention, it is not desired to limit the invention to the exact construction, dimensional relationships, and operation shown and described. Various modifications, alternative constructions, changes and equivalents will readily occur to those skilled in the art and may be employed, as suitable, without departing from the true spirit and scope of the invention. Such changes might involve alternative materials, components, structural arrangements, sizes, shapes, forms, functions, operational features or the like. The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

Aspects of the present disclosure may improve social interaction because users may indicate when they are actually available to engage in a communication session. In addition, the richness of the communicated reactions, e.g., video or audio representations of user expressions, may further improve the social interaction because such representations may convey more information with less user effort. The techniques described herein may be implemented in hardware, software, firmware, or any combination thereof. Various features described as modules, units or components may be implemented together in an integrated logic device or separately as discrete but interoperable logic devices or other hardware devices. In some cases, various features of electronic circuitry may be implemented as one or more integrated circuit devices, such as an integrated circuit chip or chipset.

If implemented in hardware, this disclosure may be directed to an apparatus such a processor or an integrated circuit device, such as an integrated circuit chip or chipset. Alternatively or additionally, if implemented in software or firmware, the techniques may be realized at least in part by a computer-readable data storage medium comprising instructions that, when executed, cause a processor to perform one or more of the methods described above. For example, the computer-readable data storage medium may store such instructions for execution by a processor.

A computer-readable medium may form part of a computer program product, which may include packaging materials. A computer-readable medium may comprise a computer data storage medium such as random access memory (RAM), read-only memory (ROM), non-volatile random access memory (NVRAM), electrically erasable programmable read-only memory (EEPROM), flash memory, magnetic or optical data storage media, and the like. In some examples, an article of manufacture may comprise one or more computer-readable storage media. In some examples, the computer-readable storage media may comprise non-transitory media. The term “non-transitory” may indicate that the storage medium is not embodied in a carrier wave or a propagated signal. In certain examples, a non-transitory storage medium may store data that can, over time, change (e.g., in RAM or cache).

The code or instructions may be software and/or firmware executed by processing circuitry including one or more processors, such as one or more digital signal processors (DSPs), general purpose microprocessors, application-specific integrated circuits (ASICs), field-programmable gate arrays (FPGAs), or other equivalent integrated or discrete logic circuitry. Accordingly, the term “processor,” as used herein may refer to any of the foregoing structure or any other structure suitable for implementation of the techniques described herein. In addition, in some aspects, functionality described in this disclosure may be provided within software modules or hardware modules.

The above disclosure is sufficient to enable one of ordinary skill in the art to practice the invention, and provides the best mode of practicing the invention presently contemplated by the inventor. While there is provided herein a full and complete disclosure of the preferred configurations of this invention, it is not desired to limit the invention to the exact construction, dimensional relationships, and operation shown and described. Various modifications, alternative constructions, changes and equivalents will readily occur to those skilled in the art and may be employed, as suitable, without departing from the true spirit and scope of the invention. Such changes might involve alternative materials, components, structural arrangements, sizes, shapes, forms, functions, operational features or the like. The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

1. A method of detecting fraudulent patient behavior in a clinical research study, the method comprising:
accessing computer-readable instructions from one or more physical memory devices for execution by one or more processors;
executing the computer-readable instructions accessed from the one or more physical memory devices by the one or more processors;
storing, in at least one of the physical memory devices, values resulting from having executed the computer-readable instructions on the one or more processors;
wherein the accessed computer-readable instructions to detect fraudulent patient behavior; and
wherein executing fraudulent patient behavior instructions further comprising:
receiving, at a computing device, a patient fingerprint image, from an imaging device of administrator or investigator computing device;
comparing, at the computing device, the received patient fingerprint image to a database of existing patient fingerprint images to determine if the patient is previously enrolled in a clinical study;
communicating a message identifying the patient may be eligible to enroll if no match is found between the received fingerprint image and the database of existing patient fingerprint images; and
performing additional analysis with respect to the patient if a match is found between the received fingerprint image and the database of existing fingerprint images.

2. The method of claim 1, wherein if no match is found between the received fingerprint image and the database of existing fingerprint images, wherein executing the fraudulent patient behavior instructions further comprising:
analyzing an existing patient electronic record to determine whether the patient is a high-risk patient; and
initially denying enrollment into the clinical study if the patient is a high-risk patient.

3. The method of claim 2, wherein executing the fraudulent patient behavior instructions further comprising: overriding, by an external computing device, the initial denial of enrollment by a sponsor administrator; and
communicating an electronic message indicating acceptance of patient's enrollment into the clinical study.

4. The method of claim 3, wherein executing the fraudulent patient behavior instructions further comprising: receiving a patient voice file and storing the patient voice file in the patient record.

5. The method of claim 4, wherein executing the fraudulent patient behavior instructions further comprising: receiving a consent document image electronically signed and storing the consent document image in the patient record.

6. The method of claim 5, wherein executing the fraudulent patient behavior instructions further comprising: receiving patient identification parameters or information;
generating a QR code, the QR code representative of the received patient identification parameters or information; and
storing the QR code in the patient record.

7. The method of claim 1, wherein the performing the additional analysis with respect to the patient if a match is found between the received fingerprint image and the database of existing fingerprint images, further comprising:
analyzing whether the patient has waited a predetermined time window after a prior clinical study; and
if the patient has not waited the predetermined time window, communicating a message to the sponsor or administrator computing device denying the patient enrollment into the clinical study.

8. The method of claim 1, wherein the performing the additional analysis with respect to the patient if a match is found between the received fingerprint image and the database of existing fingerprint images, further comprising:
analyzing whether the patient has waited a predetermined time window after a prior clinical study;
if the patient has waited the predetermined time window, analyzing whether the patient is currently enrolled in another clinical study; and
if the patient is currently enrolled in another clinical study, communicating a message to the administrator computing device denying the patient enrollment into the clinical study.

9. The method of claim 8, wherein executing the fraudulent patient behavior instructions further comprising: analyzing the patient record to determine whether the patient has been lost to follow-up for more than a set number of clinical studies during a pre-determined timeframe; and
communicating a message to the patient initially denying enrollment in the clinical study if the patient has been lost to follow-up for more than the set number of clinical studies during the predetermined timeframe.

10. The method of claim 8, wherein executing the fraudulent patient behavior instructions further comprising: analyzing the patient record to determine whether the patient has withdrawn consent more than a set number of clinical studies during a predetermined timeframe; and
communicating a message to the patient initially denying enrollment in the clinical study if the patient has withdrawn consent for more than the set number of clinical studies during the predetermined timeframe.

11. The method of claim 8, wherein executing the fraudulent patient behavior instructions further comprising: analyzing the patient record to determine if the patient has been withdrawn by an investigator, a sponsor administrator or a software developer; and
communicating a message to the patient initially denying enrollment in the clinical study if the patient has been withdrawn by the investigator, the sponsor administrator or the software developer.

12. The method of claim 8, wherein executing the fraudulent patient behavior instructions further comprising: analyzing the patient record to determine if the patient has had more than three screen failures in the last year; and
communicating a message to the patient initially denying enrollment in the clinical trial if the patient has had more than three screen failures in the last year.

13. A system to detect patient fraud in clinical trials or studies, the system comprising:
one or more memory devices, the one or more memory devices including a database to store a plurality of patient records;
one or more processors;
computer-readable instructions, the computer-readable instructions stored in the one or more memory devices in a different storage area from the database, the computer-readable instructions executable by the one or more processors to:
receive clinical study details or parameters for a clinical study;
receive a clinical study start date, a clinical study end date and clinical study comments, all for the clinical study;
create a clinical study record, the clinical study record to include the clinical study details or parameters and store the clinical study record in the database;
receive a request to add a patient to the clinical study;
verify the patient is enrollment eligible for the clinical study utilizing fingerprint validation, the fingerprint validation including comparing a received image of the patient's fingerprint to a plurality of existing patient fingerprint images in the database; and
create a patient record, the patient record to be associated with the clinical study.

14. The system of claim 13, the computer-readable instructions further executable by the one or more processors to:
if the patient is verified to be enrollment eligible, verify that the patient record does not include a high-risk indicator and allow the patient to move towards registration if the patient record does not include the high-risk indicator.

15. The system of claim 13, the computer-readable instructions further executable by the one or more processors to:
if the patient is verified to be enrollment eligible, further verity that the patient record does not include a high-risk indicator and if the patient record includes the high risk indicator, allowing the patient to move towards registration if an overriding authorization indicator or parameter is received from a sponsor administrator computing device.

16. The system of claim 13, the computer-readable instructions further executable by the one or more processors to: proceed with registration and receive registration parameters from the eligible patient.

17. The system of claim 16, the computer-readable instructions further executable by the one or more processors to: receive a patient's voice file and to store the patient's voice file in the database.

18. The system of claim 13, the computer-readable instructions further executable by the one or more processors to: receive a patient's electronically signed consent form image; and
store the patient's electronically signed consent form image in the database.

19. The system of claim 13, the computer-readable instructions further executable by the one or more processors to:
generate a QR code for the enrolled patient, the QR code including representations of a patient's parameters; and to store the QR code in the database.

20. A method of detecting fraudulent patient behavior in a clinical research study, the method comprising:
accessing computer-readable instructions from one or more physical memory devices for execution by one or more processors;
executing the computer-readable instructions accessed from the one or more physical memory devices by the one or more processors;
storing, in at least one of the physical memory devices, values resulting from having executed the computer-readable instructions on the one or more processors;
wherein the accessed computer-readable instructions to detect fraudulent patient behavior; and
wherein executing fraudulent patient behavior instructions further comprising:
receiving, at a computing device, patient parameters, from an administrator or investigator computing device;
comparing, at the computing device, the received patient parameters to a database of existing patient parameters to determine if the patient is previously enrolled in a clinical study;
communicating a message identifying the patient may be eligible to enroll if no match is found between the received patient parameters and the database of existing patient parameters; and
performing additional analysis with respect to the patient if a match is found between the received patient parameters and the database of existing fingerprint parameters.
